(19) United States
(12) Patent Application Publication
(10) Pub. No.: US 2016/0327510 A1
(43) Pub. Date: Nov. 10, 2016

(54) METHODS AND SYSTEMS FOR THE ELECTROCHEMICAL DETECTION OF ANALYTES

(71) Applicant: BOARD OF REGENTS, THE UNIVERSITY OF TEXAS SYSTEM, Austin, TX (US)

(72) Inventors: Richard M. Crooks, Austin, TX (US); Karen Scida, Austin, TX (US); Josephine Cunningham, Austin, TX (US)

(21) Appl. No.: 15/109,746

(22) PCT Filed: Dec. 19, 2014
(86) PCT No.: PCT/US14/71389

§ 371 Date: Jul. 5, 2016

Related U.S. Application Data
(60) Provisional application No. 61/923,713, filed on Jan. 5, 2014.

(51) Int. Cl. G01N 27/413; G01N 15/06; G01N 27/74; G01N 33/543; B01L 3/00

(52) U.S. Cl. CPC G01N 27/413; G01N 33/54326; B01L 3/502715; B01L 3/50273; B01L 3/502746; G01N 27/745; G01N 15/0656; B01L 2400/043; B01L 2300/0645; B01L 2300/126; B01L 2300/0887; B01L 2300/165; G01N 2015/0065

(57) ABSTRACT

Methods and devices electrochemically detect analytes. The methods employ metal particles conjugated to the analytes. The metal particles can serve as an electrochemical label for the analyte to which they are conjugated. The metal particles can be oxidized to form metal ions that can subsequently be electrochemically detected and/or quantified. The metal ions can be electrodeposited as metal on a working electrode. The potential applied at the working electrode can then be varied to reoxidize the deposited metal to metal ions. The intensity of the resulting voltammetric peak reflects the amount of metal deposited on the working electrode, and therefore the amount of metal nanoparticle label and analyte. Sensitivity can tie improved by selectively localizing the analyte-metal particle conjugate in the vicinity of the working electrode. Analytes can be detected at concentrations as low as 767 fM via anodic stripping voltammetry, with no washing steps or electrode modifications.

### STATEMENT REGARDING FEDERALLY SPONSORED RESEARCH

This invention was made with government support under Contracts No. HDTRA-1-10-1-0031 and HDTRA-1-11-1-0005 awarded by the Department of Defense/Defense Threat Reduction Agency (DTRA). The government has certain rights in this invention.

### BACKGROUND

There is a significant interest in the development of paper point-of-care (POC) devices that are cheap, user friendly, robust, sensitive, and portable. Such devices pose an effective solution to the existing economic and healthcare accessibility problems in underdeveloped countries, as well as the growing trend in more affluent societies to become better informed in terms of its health. Although commercial paper-based sensors have been around for about 25 years (e.g., pregnancy test and glucose test strips), few paper POC devices have been successfully commercialized. Such failure to produce trustworthy paper POC devices is a combination of many factors, including poor limits of detection (LOD), high non-specific adsorption (NSA), unstable reagents, long analysis time, complex user-technology interface, detection method, and poor sensitivity.

### SUMMARY

Provided herein are methods for the electrochemical detection of analytes. Methods for detecting an analyte can comprise flowing fluid along a channel to accumulate the analyte conjugated to a metal particle (i.e., an analyte conjugate) in a region of the channel in electrochemical contact with a working electrode. The channel can be, for example, a microfluidic channel. The analyte conjugate can be accumulated in the region of the channel in electrochemical contact with a working electrode by a localization element. The localization element can be any feature that is configured to increase the concentration of the analyte conjugate in the region of the channel in electrochemical contact with the working electrode in the presence of fluid flow through the channel. For example, the localization element can be a physical barrier disposed in the region of the channel in electrochemical contact with the working electrode (e.g., a material configured to physically entrap the analyte conjugate), one or more localization electrodes configured to apply an electric field to the region of the channel in electrochemical contact with the working electrode (e.g., configured to electrophoretically localize the analyte conjugate), a magnet configured to apply a magnetic field in the region of the channel in electrochemical contact with the working electrode, or a combination thereof.

Once the analyte conjugate is accumulated in the region of the channel in electrochemical contact with a working electrode, fluid flow along the channel can be interrupted. The metal particle can then be oxidized, forming a population of metal ions in the region of the channel in electrochemical contact with the working electrode. The metal particle can be oxidized by any suitable method, such as by contacting the metal particle with a suitable oxidant (e.g., permanganate or hypochlorite) or by direct electrochemical oxidation of the metal particle by a potential applied at the working electrode. In some embodiments, the localization element (e.g., the magnet) can be translocated from an incubation region, where the conjugate analyte is accumulated, to an oxidation region comprising an oxidant, thereby bringing the metal particle into contact with the oxidant. Once formed, the metal ions (and by extension the analyte) can then be electrochemically detected and/or quantified, for example, using the working electrode (e.g., by anodic stripping voltammetry).

Also provided are devices for the electrochemical detection of analytes. The devices can comprise a channel defining a path for fluid flow from a fluid inlet to a fluid outlet, a working electrode positioned in electrochemical contact with a region of the channel, and a localization element configured to accumulate the analyte conjugated to the metal particle (i.e., the analyte conjugate) in the region of the channel in electrochemical contact with the working electrode.

As described above, the localization element can be any feature that is configured to increase the concentration of the analyte conjugate in the region of the channel in electrochemical contact with the working electrode in the presence of fluid flow through the channel. For example, the localization element can be a physical barrier disposed in the region of the channel in electrochemical contact with the working electrode, one or more localization electrodes configured to apply an electric field to the region of the channel in electrochemical contact with the working electrode, a magnet configured to apply a magnetic field in the region of the channel in electrochemical contact with the working electrode, or a combination thereof.

In certain embodiments, the localization element can comprise a magnet configured to apply a magnetic field in the region of the channel in electrochemical contact with the working electrode. In these embodiments, the devices can comprise a channel defining a path for fluid flow from a fluid inlet to a fluid outlet, an electrode positioned in electrochemical contact with a region of the channel, and a magnet configured to apply a magnetic field to the region of the channel positioned in electrochemical contact with the electrode.

Devices can further include a counter electrode, a reference electrode, or combinations thereof in electrochemical contact with the channel. Optionally, if desired for a particular detection method, the devices can further include a second working electrode positioned in electrochemical contact with a second region of the channel, and a second localization element configured to accumulate an analyte conjugated to a metal particle (i.e., an analyte conjugate) in the second region of the channel in electrochemical contact with the second working electrode.

Devices can further comprise an engageable platform that can be translocated from a retracted position to a deployed position. When the engageable platform is in the retracted position, the engageable platform is fluidly independent from the channel. When the engageable platform is in the deployed position, the engageable platform is in fluid contact with the region of the channel in electrochemical contact with the working electrode. An oxidant, such as potassium permanganate or hypochlorite, can be disposed on the engageable platform. In these embodiments, the oxidant can be introduced to the region of the channel in electrochemical contact with the working electrode by translocation of the engageable platform (e.g., to oxidize the metal particle).

The devices and methods described herein are inexpensive, user friendly (they employ electrochemical detection without any washing steps or electrode modification), sensitive, portable, robust (they employ metal particles for signal amplification as opposed to enzymes), efficient, rapid (completion of analysis in 4.6 min), and can detect low concentrations (767 fM). As such, the device and methods are well suited for use in numerous applications including point-of-care (POC) diagnostics.

### DESCRIPTION OF FIGURES

FIG. 1 is a schematic side view of the four layers of a device for the electrochemical detection of analytes as viewed along the axis of fluid flow.

FIG. 2 displays a schematic side view of the assembled device for the electrochemical detection of analytes as viewed along the axis of fluid flow.

FIG. 3 displays a schematic top view of the four layers of the device for the electrochemical detection of analytes.

FIG. 4 displays a schematic top view of the device for the electrochemical detection of analytes, with the first layer aligned with the second layer.

FIG. 5 displays a schematic top view of the device for the electrochemical detection of analytes, with the first layer aligned with the second layer and the third layer in (a) position 1 and (b) position 2.

FIG. 6 displays a schematic top view of the device for the electrochemical detection of analytes, with all four layers aligned (a) position 1 and (b) position 2.

FIG. 7 displays a cutaway side view of the device for the electrochemical detection of analytes with all four layers aligned in position 1 as viewed perpendicular to the axis of fluid flow.

FIG. 8 displays a side view of the device for the electrochemical detection of analytes with all four layers aligned in position 2 as viewed perpendicular to the axis of fluid flow.

FIG. 9 is a schematic illustration of a method for the electrochemical detection of an analyte. In this embodiment, a sandwich-type assay is used to detect the analyte. In this case, an analyte is bound to a first antibody and a second antibody. A metal particle (e.g., a silver nanoparticle) is bound to the first antibody and a magnetic particle (e.g., a magnetic microbead) is bound to the second antibody. The analyte conjugated to the metal particle and the magnetic particle is flowed along a channel (step a), and accumulated by an applied magnetic field in a region of the channel in electrochemical contact with a working electrode. Once accumulated, the metal particles are contacted with an oxidant, and oxidized to form metal ions (step b). The metal ions are then electrochemically deposited, on the working electrode (e.g., by holding the working electrode at a reducing potential; step c), and detected by electrochemically oxidizing the deposited Ag to Ag⁺ (e.g., by sweeping the potential of the electrode positive to obtain an anodic current transient; step d). The charge under the current-time transient reflects the number of metal ions present in the channel (and by extension the concentration of the analyte). As shown in step a′, in the absence of the analyte, the metal nanoparticles bound to the first antibody flow in the channel without accumulating at the working electrode, and no signal for the analyte is observed.

FIG. 10 is a schematic illustration of a method for the electrochemical detection of an analyte. In this embodiment, competitive binding is used to detect a molecule of interest. An analyte (e.g., a small molecule such as estradiol) bound to a metal particle (e.g., a silver nanoparticle), an antibody for the analyte bound to a magnetic particle (e.g., a magnetic microbead), and a molecule of interest (e.g., estradiol) that competitively binds with the antibody are flowed along a channel. A magnetic field is applied to a region of the channel in electrochemical contact with a working electrode to accumulate the metal particles in the region of the channel in electrochemical contact with the working electrode (steps 1a). In the absence of the molecule of interest, the analyte conjugated to the metal particle and the magnetic particle is accumulated by an applied magnetic field in a region of the channel in electrochemical contact with a working electrode (step 1a, top). In the presence of the molecule of interest, the molecule of interest and the analyte bound to the metal particle competitively bind to the antibody bound to a magnetic particle. In this case, a portion of the molecule of interest remains bound to the metal particle in the region of the channel in electrochemical contact with a working electrode, and a portion of the analyte bound to the metal particle flows downstream from the region of the channel in electrochemical contact with the working electrode (step 1a, bottom). Next the metal particles remaining in the region of the channel in electrochemical contact with the working electrode are contacted with an oxidant, and oxidized to form metal ions (step 1b). The metal ions are then electrochemically detected as described above (steps 1c and 1d) to detect and/or quantify the molecule of interest. As the concentration of the molecule of interest increases, one would expect to observe a decreased electrochemical signal at the working electrode. A control experiment can be simultaneously performed in the same channel using a metal particle-magnetic particle conjugate that does not competitively bind with the molecule of interest (steps 2a-2d). This control experiment should always provide an electrochemical signal to confirm the test was successfully performed. This embodiment can be performed using the device illustrated in FIG. 27.

FIG. 11 is a schematic illustration of a method for the electrochemical detection of a polynucleotide (e.g., DNA). In this embodiment, an analyte (e.g., a polynucleotide such as a single strand of DNA) is contacted with a first recognition element (e.g., a first polynucleotide probe having a complementary sequence to a first portion of the analyte) and a second recognition element (e.g., a second polynucleotide probe having a complementary sequence to a second portion of the analyte). A metal particle (e.g., a silver nanoparticle) is bound to the first recognition element and a magnetic particle (e.g., a magnetic microbead) is bound to the second recognition element. The analyte can then be electrochemically detected using the method illustrated in FIG. 9. Briefly, the analyte conjugated to the metal particle and the magnetic particle can be flowed along a channel (step a), and accumulated by an applied magnetic field in a region of the channel in electrochemical contact with a working electrode. Once accumulated, the metal particles are contacted with an oxidant, and oxidized to form metal ions (step b). The metal ions can then be electrochemically detected (steps 1c and 1d) to detect and/or quantify the analyte.

FIG. 12 is a schematic illustration of a method for the electrochemical detection of an analyte. In this embodiment, a surrogate conjugated to a fixed support is used to detect a molecule of interest. A fixed analyte support (e.g., an aptamer that specifically binds the molecule of interest) is immobilized on a surface (e.g., on a porous hydrophilic substrate, such as paper, present in the fluid inlet of a device). A surrogate (e.g., a recognition element for the aptamer such as a polynucleotide probe having a complementary sequence to a portion of the aptamer) is bound to the fixed analyte support. A metal nanoparticle (e.g., a silver nanoparticle) is bound to the surrogate. The surrogate-fixed analyte support conjugate is contacted with the molecule of interest. The molecule of interest binds to the fixed analyte support, displacing the surrogate bound to a metal nanoparticle. Once displaced from the fixed analyte support, the surrogate bound to a metal nanoparticle is contacted with and binds to a recognition element for the surrogate (e.g., a polynucleotide having a complementary sequence to a portion of the analyte). The recognition element for the surrogate is bound to a magnetic particle. The surrogate can then be electrochemically detected using the method illustrated in FIG. 9. Briefly, the surrogate conjugated to the metal particle and the magnetic particle can be flowed along a channel (step a), and accumulated by an applied magnetic field in a region of the channel in electrochemical contact with a working electrode. Once accumulated, the metal particles are contacted with an oxidant, and oxidized to form metal ions (step b). The metal ions can then be electrochemically detected (steps 1e and 1d) to detect and/or quantify the surrogate. Because the concentration of the surrogate is proportional to the concentration of the molecule of interest, the molecule of interest can be detected and/or quantified by extension.

FIG. 13 is a schematic illustration of a method for the simultaneous electrochemical detection of multiple analytes. In the example embodiment illustrated in FIG. 13, three analytes are simultaneously detected and/or quantified. The first analyte (molecule 1) is bound to a first antibody and a second antibody. A first metal particle (comprising metal 1) is bound to the first antibody and a magnetic particle (e.g., a magnetic microbead) is bound to the second antibody. The second analyte (molecule 2) is bound to a third antibody and a fourth antibody. A second metal particle (comprising metal 2) is bound to the third antibody and a magnetic particle (e.g., a magnetic microbead) is bound to the fourth antibody. The third analyte (molecule 3) is bound to a fifth antibody and a sixth antibody. A third metal particle (comprising metal 3) is bound to the fifth antibody and a magnetic particle (e.g., a magnetic microbead) is bound to the sixth antibody. Metal 1, metal 2, and metal 3 are selected to be different and possess distinct reduction potentials, such that the electrochemical signals from metal 1, metal 2, and metal 3 can be individually resolved at the working electrode, as discussed in more detail below. The analytes can then be electrochemically detected using the method illustrated in FIG. 9. The analytes, each conjugated to the metal particle and the magnetic particle are flowed along a channel (step a), and accumulated by an applied magnetic field in a region of the channel in electrochemical contact with a working electrode. Once accumulated, the metal particles (metal particle 1, metal particle 2, and/or metal particle 3) are contacted with an oxidant, and oxidized to form metal ions (step b). The metal ions can then be electrochemically detected (steps 1c and 1d) to detect and/or quantify the first analyte, the second analyte, the third analyte, or combinations thereof. As illustrated in the hypothetical plots, because metal 1, metal 2, and metal 3 are selected to be different and possess distinct reduction potentials, the electrochemical signals from metal 1, metal 2, and metal 3 can be individually resolved at the working electrode. This allows for the simultaneous detection and/or quantification of multiple analytes.

FIG. 14 displays micrograph images showing the surface of the glassy carbon working electrode (GCE, 1.0 mm in diameter) after bulk solution-based electrodeposition and stripping steps in the presence of 50.0 μL of KMnO₄, 50.0 μL of deionized water, and 125.0 μL of 100.0 mM phosphate buffer (PB) containing 100.0 mM NaCl. (a) GCE surface before experiment at 5.0 mM KMnO₄. (b) GCE surface before experiment at 187.0 μM KMnO₄. (c) GCE surface after experiment at 5.0 mM KMnO₄. (d) GCE surface after experiment at 187.0 μM KMnO₄. (e) Resulting anodic stripping voltammetry of 50.0 μL of 75.0 pM citrate-capped AgNPs, 125.0 μL of 100.0 mM PB containing 100.0 mM NaCl, and 50.0 μL of KMnO₄ at 5.0 mM and 187.0 μM.

FIG. 15 displays the effect of KMnO₄ reduction on the electrochemical signal in bulk solution with two different setups. (a) Setup in which the glassy carbon electrode (GCE, 1.0 mm in diameter) is facing up. (b) Setup in which the GCE is facing down. (c) Stripping waves showing experiments performed in triplicate with the WE facing up (214±9 nC) and facing down (211±7 nC).

FIG. 16 displays the oSlip and stencil design and dimensions.

FIG. 17 displays UV-Vis spectra showing the formation of the AgNP/biotin/streptavidin/magnetic microbead composite. The trace labeled “AgNP/biotin” corresponds to the biotinylated AgNP absorbance before incubation with streptavidin-coated magnetic microbeads. The trace labeled “Supernatant” corresponds to the supernatant absorbance after incubating biotinylated AgNPs with streptavidin-coated magnetic microbeads.

FIG. 18 displays the oSlip and bulk solution plot of charge under the stripping peak as a function of KMnO₄ moles added. The number of Ag moles was kept constant. The error bars on each data point represent the standard deviation of three different measurements.

FIG. 19 displays a calibration curve of charge recorded under each peak as a function of citrate-capped AgNP concentration present in bulk solution. The error bars on each data point represent the standard deviation of three different measurements. Inset: Stripping waves of citrate-capped AgNP concentrations in the linear range (3.3 to 25 pM).

FIG. 20 displays images showing the citrate-capped AgNP size distribution. (a) Results in the absence of KMnO₄. A Gaussian distribution centered at 20 nm was recorded. (b) Results in the presence of 5.6 nmoles of KMnO₄. A Gaussian distribution centered at 12 nm was recorded. Each experiment was performed in triplicate. The x-axis is the AgNP diameter in nm. The y-axis is the AgNP concentration in particles/mL.

FIG. 21 displays the electrochemical signal in the presence and absence of 100.0 mM NaCl. Charge with 100.0 mM NaCl and without NaCl is 200±29 nC and 187±18 nC, respectively. Both experiments were performed a total of three times.

FIG. 22 displays a time dependent study of KMnO₄ resolvation in the oSlip. The error bars on each data point represent the standard deviation of three different oSlips. Inset: Cyclic voltammograms of KMnO₄ initiated 12 s after slipping Layer 3 into position 2 corresponding to three different oSlips. The Ohmic drop compensation for a waiting time of 5 s was pre-set to 4.0 kΩ.

FIG. 23 displays a schematic of an example device.

FIG. 24 displays fluorescence micrographs showing the placement of 2.8 μm in diameter fluorescein-modified magnetic microbeads on the working electrode (WE) of a paper device for the Control (a) and Test (b) experiments. The dashed lines represent the location of the WE. The solid lines show the areas used to measure the fluorescein-modified magnetic microbeads fluorescence intensity. Equivalent areas were used to measure the fluorescence intensity of both images. Each experiment was background corrected and performed in triplicate.

FIG. 25 displays a schematic diagram of an electrochemical system.

FIG. 26 displays the electrochemical response of the oSlip proof-of-concept experiment. (a) Stripping peaks at different AgNP concentrations (contained in the composite) present in 50.0 μL of 100.0 mM PB containing 100.0 mM NaCl. Inset shows the stripping peaks corresponding to the background and AgNP concentrations of 767 fM and 1.5 pM. (b) Calibration curve of charge under each stripping peak as a function of the AgNP concentration present in the composite. The linear fit equation is y=1.035*10⁻⁷×−8.742*10⁻⁸. Inset shows the linear range and two extra data points (55.3 and 110.5 pM) demonstrating where the data deviates from linearity Ohmic drop compensation was performed for each oSlip before starting the experiment (4170±360Ω, for 15 devices). The error bars of each data point represent the standard deviation of the signal obtained with three different oSlips.

FIG. 27 illustrates a device for the electrochemical detection of an analyte that includes a control assay in the same channel. The device can be used in practicing the methods schematically illustrated in FIGS. 9, 10, 11, and 12. The device includes four layers as in the embodiment illustrated in FIG. 23. However, the device includes four electrodes: a first working electrode (a; analyte working electrode), a second working electrode (b; control working electrode), a reference electrode (c), and a counter electrode (d). The device includes a first magnet aligned with the first working electrode so as to apply a magnetic field within the region of the channel in the second layer in electrochemical contact with the first working electrode, and a second magnet aligned with the second working electrode so as to apply a magnetic field within the region of the channel in the second layer in electrochemical contact with the second working electrode. The fluid inlet comprises a porous hydrophilic substrate, such as paper, onto which reagents for the detection of the molecule of interest (e.g., an analyte bound to a metal particle and an antibody for the analyte bound to a magnetic particle in the case of the method schematically illustrated in FIG. 10) can be deposited. The fluid inlet comprises a hydrophilic barrier such that the two reagents can be isolated from one another prior to being contacted with the molecule of interest. The device also includes a control platform in fluid contact with the channel in the second layer downstream of the first working electrode but upstream of the second working electrode (illustrated as a rectangle between electrode a and b in FIG. 23). A control complex (e.g., a metal particle-magnetic particle conjugate that does not competitively bind with the molecule of interest in the case of the method schematically illustrated in FIG. 10) can be deposited on the control platform. When a fluid comprising the molecule of interest is applied to the fluid inlet, the assay for the detection of the analyte is performed, as described above, at the first working electrode. The fluid flowing through the channel also draws the control complex from the control platform. The control complex is flowed along the channel, and accumulated by an applied magnetic field in a region of the channel in electrochemical contact with the second electrode (providing for the control experiment).

FIG. 28 illustrates a device for the electrochemical detection of analytes. The device includes four layers as in the embodiment illustrated in FIG. 23. However, the third layer of the device can be translocated between three positions. In position 1 (initial and incubation position, panel c), a hydrophobic region of layer three fluidly isolates the channel in the second layer from the channel in the fourth layer. In this embodiment, fluid can be added to the fluid inlet; however, fluid flow through the channel does not commence. The fluid inlet comprises a porous hydrophilic substrate, such as paper, onto which reagents for the detection of the molecule of interest (which can be, for example a first antibody bound to a metal nanoparticle and a second antibody bound to a magnetic particle in the case of the method schematically illustrated in FIG. 9) can be deposited. A fluid sample comprising the molecule of interest (e.g., the analyte in the case of the method schematically illustrated in FIG. 9) can then be applied to the fluid inlet where it contacts the reagents and is incubated. Following incubation, the third layer can be translocated to position 2 (flow position, panel c) allowing fluid to flow to the fluid outlet of the device. Once flow is complete, the third layer can be translocated to position 3 (deployed position, panel c), bringing an engageable platform comprising an oxidant into fluid contact with the channel in second layer.

### DETAILED DESCRIPTION

The methods and devices described herein may be understood more readily by reference to the following detailed description of specific aspects of the disclosed subject matter, figures and the examples included therein.

Before the present devices and methods are disclosed and described, it is to be understood that the aspects described below are not intended to be scope by the specific devices and methods described herein, which are intended as illustrations. Various modifications of the devices and methods in addition to those shown and described herein are intended to fall within the scope of that described herein. Further, while only certain representative devices and method steps disclosed herein are specifically described, other combinations of the devices and method steps also are intended to fall within the scope of that described herein, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Methods

Provided herein are methods for the electrochemical detection of analytes. The methods employ metal particles (e.g., metal nanoparticles) conjugated to analytes. The metal particles can serve as an electrochemical label for the analyte to which they are conjugated. Specifically, the metal particles can be oxidized to form metal ions that can subsequently be electrochemically detected and/or quantified. For example, the metal ions can be electrodeposited as metal on a working electrode. The potential applied at the working electrode can then be varied to reoxidize the deposited metal to metal ions. The intensity of the resulting voltammetric peak reflects the amount of metal deposited on the working electrode, and therefore the amount of metal nanoparticle label (and by extension analyte) present in a sample. Sensitivity can be improved by selectively localizing the analyte-metal particle conjugate in the vicinity of the working electrode. Using this method, analytes can be detected at concentrations as low as 767 fM via anodic stripping voltammetry, with no washing steps or electrode modifications.

Methods for detecting an analyte can comprise flowing fluid along a channel to accumulate the analyte conjugated to a metal particle (i.e., an analyte conjugate) in a region of the channel in electrochemical contact with a working electrode. The channel can be, for example, a microfluidic channel. The analyte conjugate can be accumulated in the region of the channel in electrochemical contact with a working electrode by a localization element. The localization element can be any feature that is configured to increase the concentration of the analyte conjugate in the region of the channel in electrochemical contact with the working electrode in the presence of fluid flow through the channel. For example, the localization element can be a physical barrier disposed in the region of the channel in electrochemical contact with the working electrode (e.g., a material configured to physically entrap the analyte conjugate), one or more localization electrodes configured to apply an electric field to the region of the channel in electrochemical contact with the working electrode (e.g., configured to electrophoretically localize the analyte conjugate), a magnet configured to apply a magnetic field in the region of the channel in electrochemical contact with the working electrode, or a combination thereof.

Once the analyte conjugate is accumulated in the region of the channel in electrochemical contact with a working electrode, fluid flow along the channel can be interrupted. The metal particle can then be oxidized, forming a population of metal ions in the region of the channel in electrochemical contact with the working electrode. The metal particle can be oxidized by any suitable method, such as by contacting the metal particle with a suitable oxidant or by direct electrochemical oxidation of the metal particle by a potential applied at the working electrode. Once formed, the metal ions (and by extension the analyte) can then be electrochemically detected and/or quantified, for example, using the working electrode.

The analyte can be, for example, an antibody, peptide (natural, modified, or chemically synthesized), protein (e.g., a glycoprotein, a lipoprotein, or a recombinant protein), polynucleotide (e.g, DNA or RNA, an oligonucleotide, an aptamer, or a DNAzyme), lipid, polysaccharide, small molecule organic compound (e.g., a hormone, a prohormone, a narcotic, or a small molecule pharmaceutical), pathogen (e.g., bacteria, virus, or fungi, or protozoa), or combination thereof.

In some embodiments, the analyte can be a molecule of interest present in a fluid sample that is introduced into the channel. By way of example, the fluid sample can be a bodily fluid. “Bodily fluid”, as used herein, refers to a fluid composition obtained from or located within a human or animal subject. Bodily fluids include, but are not limited to, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions, as well as mixtures containing homogenized solid material, such as feces, tissues, and biopsy samples. The molecule of interest can be, for example, a biomarker (i.e., a molecular indicator associated with a particular pathological or physiological state) present in the bodily fluid that can be assayed to identify risk for, diagnosis of, or progression of a pathological or physiological process in a subject. Examples of biomarkers include proteins, hormones, prohormones, lipids, carbohydrates, DNA, RNA, and combinations thereof.

When the analyte is a molecule of interest present in the fluid sample that is introduced into the channel, methods can further involve conjugating the molecule of interest to a metal particle to form an analyte complex (e.g., for example by contacting the molecule of interest with a metal nanoparticle bound to a recognition element for the molecule of interest, as described in more detail below). Conjugation can occur in the fluid sample prior to introduction into the channel, such that the resulting analyte complex is introduced into the channel. Alternatively, conjugation can occur in situ within the device (e.g., by contacting with by contacting the molecule of interest with a metal nanoparticle bound to a recognition element that is deposited on or within the channel or a fluid inlet fluidly connected thereto).

In other embodiments, the analyte can be a surrogate for the molecule of interest. The surrogate can be an analyte whose concentration in the fluid flowing through the channel is proportional to the concentration of the molecule of interest in the fluid sample, such that by detecting and/or quantifying the surrogate using the electrochemical methods described herein, the molecule of interest can be detected and/or quantified. By way of example, a fixed analyte support (e.g., an aptamer that specifically binds a molecule of interest) can immobilized on or within the channel or a fluid inlet fluidly connected thereto. A surrogate (e.g., a recognition element for the aptamer such as a polynucleotide probe having a complementary sequence to a portion of the aptamer) can be bound to the fixed analyte support. When the surrogate-fixed analyte support conjugate is contacted with the molecule of interest, the molecule of interest binds to the fixed analyte support, displacing the surrogate. The surrogate then functions as the analyte in the detection methods described above.

The metal particle can be, for example, a metal nanoparticle. The metal particle comprise any suitable metal, such as gold, silver, copper, platinum, rhodium, palladium, iridium, nickel, iron, bismuth, cadmium, cobalt, or combinations thereof. The metal particle can also comprise a suitable metal compound, such as, for example, a metal oxide, halide, and/or chalcogenide, such as Ag₂O, AgI, Bi₂O₅, CuO, Cd₃P₂, CdS, CdSe, CdTe, Co₂O₃, CrO₃, Cu₂S, HgI₂, MnO₂, PbS, PbO₂, SnO₂, TiO₂, RuO₂, ZnO, ZnS or ZnO₂. Suitable metal particles can be selected in view of a number of factors, including the nature of the oxidation process employed, the presence or absence of other species present in the fluid sample flowing through the channel, the nature of the electrochemical techniques employed, the desired stability of the metal particle towards environmental conditions (e.g., stability in air), compatibility with a desired means of conjugation to the analyte, and combinations thereof. For example, in some embodiments, the metal particle can be formed from a metal or metal compound that is not present (or is only present at low levels) in the fluid sample flowing through the channel. In some cases, the metal particle can be selected such that it can be reduced by an oxidant (e.g., the metal particle can be selected such that it has a reduction potential that is more negative than the oxidant).

The analyte can be conjugated to the metal particle by any suitable covalent or non-covalent means. In some embodiments, the analyte can be bound to the metal particle by a recognition element. For example, the metal particle can be bound (via any non-covalent or covalent means) to a recognition element for the analyte, which can be bound to the analyte.

Recognition elements for particular analytes are known in the art. An appropriate recognition element for the formation of an analyte conjugate can be selected in view of a number of considerations including analyte identity, analyte concentration, and the nature of the sample in which the analyte is to be bound. Suitable recognition elements include antibodies, antibody fragments, antibody mimetics (e.g., engineered affinity ligands such as AFFIBODY® affinity ligands), peptides (natural or modified peptides), proteins (e.g., recombinant proteins, host proteins), polynucleotides (e.g, DNA or RNA, oligonucleotides, aptamers, or DNAzymes), receptors, ligands, antigens, organic small molecules (e.g., antigen or enzymatic co-factors), and combinations thereof.

In some embodiments, the recognition element selectively associates with the analyte. The term “selectively associates”, as used herein when referring to a recognition element, refers to a binding reaction which is determinative for the analyte in a heterogeneous population of other similar compounds. Generally, the interaction is dependent upon the presence of a particular structure (e.g., an antigenic determinant or epitope) on the binding partner. By way of example, an antibody or antibody fragment selectively associates to its particular target (e.g., an antibody specifically binds to an antigen) but it does not bind in a significant amount to other proteins present in the sample or to other proteins to which the antibody may come in contact in an organism.

In some embodiments, a recognition element can be a molecule that has an affinity constant (Ka) greater than about 10⁵ M⁻¹ (e.g., greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, greater than about 10¹² M⁻¹, or more) with that analyte.

In certain embodiments, the recognition element comprises an antibody. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen. The term encompasses intact and/or full length immunoglobulins of types IgA, IgG (e.g., IgG1, IgG2, IgG3, IgG4), IgE, IgD, IgM, IgY, antigen-binding fragments and/or single chains of complete immunoglobulins (e.g., single chain antibodies, Fab fragments, F(ab′)2 fragments, Fd fragments, scFv (single-chain variable), and single-domain antibody (sdAb) fragments), and other proteins that include at least one antigen-binding immunoglobulin variable region, e.g., a protein that comprises an immunoglobulin variable region, e.g., a heavy (H) chain variable region (VH) and optionally a light (L) chain variable region (VL). The light chains of an antibody may be of type kappa or lambda.

An antibody may be polyclonal or monoclonal. A polyclonal antibody contains immunoglobulin molecules that differ in sequence of their complementarity determining regions (CDRs) and, therefore, typically recognize different epitopes of an antigen. Often a polyclonal antibody is derived from multiple different B cell lines each producing an antibody with a different specificity. A polyclonal antibody may be composed largely of several subpopulations of antibodies, each of which is derived from an individual B cell line. A monoclonal antibody is composed of individual immunoglobulin molecules that comprise CDRs with the same sequence, and, therefore, recognize the same epitope (i.e., the antibody is monospecific). Often a monoclonal antibody is derived from a single B cell line or hybridoma. An antibody may be a “humanized” antibody in which for example, a variable domain of rodent origin is fused to a constant domain of human origin or in which some or all of the complementarity-determining region amino acids often along with one or more framework amino acids are “grafted” from a rodent, e.g., murine, antibody to a human antibody, thus retaining the specificity of the rodent antibody.

An appropriate analyte conjugate and localization element can be selected in combination so as to facilitate accumulation of the analyte conjugate in the region of the channel in electrochemical contact with a working electrode. For example, in some embodiments, the analyte conjugate is charged (e.g., the analyte itself is charged, the metal particle is charged, or the analyte and/or the metal particle is conjugated to a charged moiety such as a charged molecule or charged particle), and the localization element comprises a localization electrode configured to apply an electric field to the region of the channel, so as to increase the concentration of the charged analyte conjugate in the region of the channel in electrochemical contact with the working electrode. In these embodiments, methods of detecting the analyte can comprise flowing fluid comprising the charged analyte conjugated to the metal particle along the channel, and applying electric field via one or more localization electrodes to accumulate the charged analyte conjugated to the metal particle in the region of the channel in electrochemical contact with a working electrode.

In some embodiments, the localization element can comprise a physical barrier disposed in the region of the channel in electrochemical contact with the working electrode. The physical barrier can be any suitable material configured to physically entrap the analyte conjugate. For example, the physical barrier can be a porous hydrophilic material (e.g., paper) or a matrix of polymer beads disposed within the fluid flow path formed by the channel that can physically entrap the analyte conjugate. In some embodiments, the analyte conjugate can further include a steric particle (e.g., a microbead) conjugated to the analyte and/or the metal particle to increase the hydrodynamic volume of the analyte, thereby facilitating entrapment of the analyte conjugate in the physical barrier. In these embodiments, methods of detecting the analyte can comprise flowing fluid comprising the analyte conjugated to the metal particle along the channel to contact the physical barrier such that the analyte accumulates in the region of the channel in electrochemical contact with a working electrode.

In certain embodiments, the localization element can comprise a magnet configured to apply a magnetic field in the region of the channel in electrochemical contact with the working electrode, and the analyte conjugate can comprise a magnetic moiety. For example, the analyte conjugate can comprise an analyte conjugated to a metal particle and a magnetic particle.

The magnetic particle can be any magnetic particle that can be conjugated to the analyte and which can provide for localization of the bound analyte under an applied magnetic field. For example, the magnetic particle can be a magnetic microbead. Magnetic microbeads are superparamagnetic, monodisperse, polymer beads that comprise a dispersion of a magnetic material (e.g., gamma Fe₂O₃ and Fe₃O₄) throughout the polymer bead. The microbeads are coated with a thin polymer shell which encases the magnetic material and provides a defined surface area for the adsorption or coupling of various molecules. Suitable magnetic microbeads are known in the art, and are commercially available from Life Technologies under the tradename DYNABEADS®.

The analyte can be conjugated to the magnetic particle by any suitable covalent or non-covalent means. In some embodiments, the analyte can be bound to the magnetic particle by a recognition element, as described above. For example, the magnetic particle can be bound (via any non-covalent or covalent means) to a recognition element for the analyte that can be bound to the analyte.

In certain embodiments, the localization element can comprise a magnet configured to apply a magnetic field in the region of the channel in electrochemical contact with the working electrode, and the analyte conjugate can comprise an analyte bound to a first antibody and a second antibody, wherein a metal particle is bound to the first antibody and a magnetic particle is bound to the second antibody.

In certain embodiments, the localization element can comprise a magnet configured to apply a magnetic field in the region of the channel in electrochemical contact with the working electrode, and the analyte conjugate can comprise an analyte bound to a first polynucleotide and a second polynucleotide, wherein a metal particle is bound to the first polynucleotide and a magnetic particle is bound to the second polynucleotide. In some of these embodiments, the analyte comprises a polynucleotide.

In these embodiments, methods of detecting the analyte can comprise flowing fluid comprising the analyte conjugated to the metal particle and the magnetic particle along the channel, and applying the magnetic field to accumulate the analyte conjugated to the metal particle and the magnetic particle in the region of the channel.

As described above, once the analyte conjugate is accumulated in the region of the channel in electrochemical contact with a working electrode, the metal particle can then be oxidized to form a population of metal ions in the region of the channel in electrochemical contact with the working electrode. The metal particle can be oxidized by any suitable oxidation method, such as by contacting the metal particle with a suitable oxidant or by direct electrochemical oxidation of the metal particle by a potential applied at the working electrode. In certain embodiments, oxidizing the metal particle comprises contacting the metal particle with a suitable oxidant. In certain embodiments, the region of the channel in electrochemical contact with the working electrode can comprise an incubation region and an oxidation region, wherein the oxidation region can comprise an oxidant. In these embodiments, the localization element (e.g., a magnet) can be used to accumulate the analyte conjugate in the incubation region, then the localization element can be translocated from the incubation region to the oxidation region thereby bringing the metal particle into contact with the oxidant. The oxidant can be any suitable oxidant known in the art. Examples of oxidants include, but are not limited to, nitric acid, sulfuric acid, peroxides (e.g., hydrogen peroxide), peroxydisulfuric acid, peroxymonosulfuric acid, halogen compounds (e.g., chlorite, chlorate, perchlorate), hypohalite compounds (e.g., hypochlorites such as sodium hypochlorite), hexavalent chromium compounds (e.g., chromate and dichromate salts), permanganate compounds (e.g. potassium permanganate), sodium perborate, nitrous oxide, silver oxide, osmium tetroxide, cerium(IV) oxide, potassium nitrate, and combinations thereof. In some embodiments, the oxidant comprises potassium permanganate. In other embodiments, the oxidant comprises an oxidant that is electrogenerated in situ in the channel. A suitable oxidant can be selected in view of a number of factors, including the desired stability of the oxidant towards environmental conditions (e.g., stability in air) and the composition of the metal particle to be oxidized. For example, the oxidant can be selected such that it can effectively oxidize the metal particle to produce a population of metal ions (e.g., the oxidant can be selected such that it has a reduction potential that is more positive than the metal particle).

Once formed, the metal ions (and by extension the analyte) can then be electrochemically detected and/or quantified, for example, using the working electrode. Various techniques of electrochemical analysis may be used to assay the dissolved metal ions. They are preferentially anodic stripping voltammetry with a potential scan which may be linear, cyclic, square-wave, normal pulse or differential pulse, or with a superimposed sinusoidal voltage, or else anodic stripping chronopotentiometry. However, other techniques may be used, such as ion exchange voltammetry, adsorptive cathodic stripping voltammetry (or polarography) with a scan which may be linear, cyclic, square-ware, normal pulse or differential pulse, or with a superimposed sinusoidal voltage, or else chronoamperometry, chronocoulometry or linear, cyclic, square-wave, normal pulse or differential pulse voltammetry (or polarography) or voltammetry (or polarography) with a superimposed sinusoidal voltage. These techniques require a possibly two-electrode or even three-electrode assembly, e.g., an assembly comprising the working electrode, a reference electrode, and a counter electrode.

A variety of potential assays (e.g., sandwich-type assays, competitive binding assays, etc.) can be envisioned that employ the electrochemical detection methods described above for analyte detection and/or quantification. The precise design of such assays will vary based on, for example, the nature of the analyte and the localization element used. By way of example, several methods of detection that employ magnetic localization are described below. While these methods include particular method steps, components, and analyte conjugates based on the use of a magnetic localization (e.g., an analyte conjugate comprising an analyte conjugated to a metal particle and a magnetic particle), it will be understood that these methods can be adapted to employ method steps, components, and analyte conjugates based on the use of an alternative localization element.

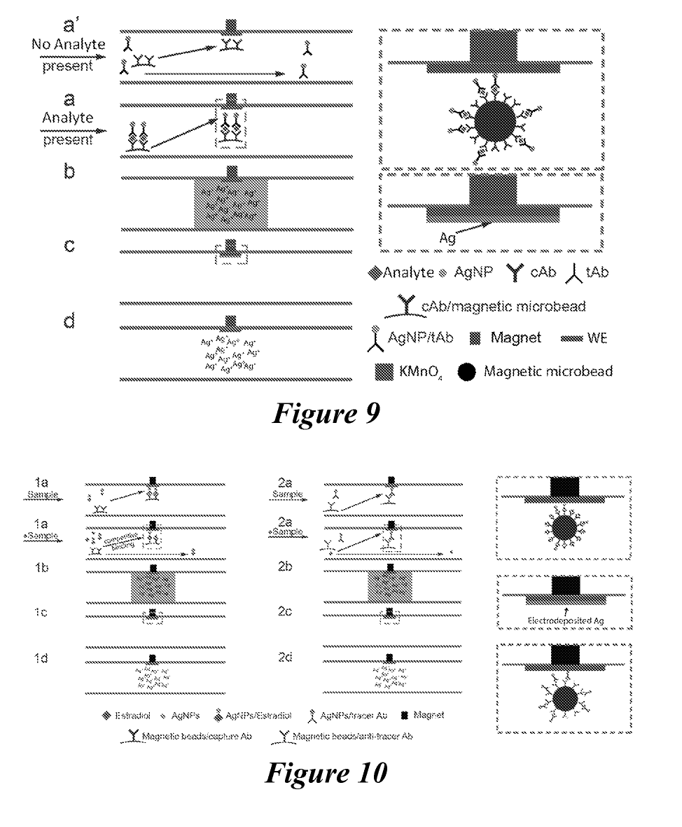

An example method for the electrochemical detection of an analyte is schematically illustrated in FIG. 9. In this embodiment, a sandwich-type assay is used to detect the analyte. In this case, the analyte (e.g., a protein such as ricin) is bound to a first antibody and a second antibody. A metal particle (e.g., a silver nanoparticle) is bound to the first antibody and a magnetic particle (e.g., a magnetic microbead) is bound to the second antibody. The analyte conjugated to the metal particle and the magnetic particle is flowed along a channel (step a), and accumulated by an applied magnetic field in a region of the channel in electrochemical contact with a working electrode. Once accumulated, the metal particles are contacted with an oxidant, and oxidized to form metal ions (step b). The metal ions are then electrochemically deposited, on the working electrode (e.g., by holding the working electrode at a reducing potential; step c), and detected by electrochemically oxidizing the deposited Ag to Ag⁺ (e.g., by sweeping the potential of the electrode positive to obtain an anodic current transient; step d). The charge under the current-time transient reflects the number of metal ions present in the channel (and by extension the concentration of the analyte). As shown in step a′, in the absence of the analyte, the metal nanoparticles bound to the first antibody flow in the channel without accumulating at the working electrode, and no signal for the analyte is observed.

An example of another method for the electrochemical detection of an analyte is schematically illustrated in FIG. 10. In this embodiment, competitive binding is used to detect a molecule of interest. An analyte (e.g., a small molecule such as estradiol) bound to a metal particle (e.g., a silver nanoparticle), an antibody for the analyte bound to a magnetic particle (e.g., a magnetic microbead), and a molecule of interest (e.g., estradiol) that competitively binds with the antibody are flowed along a channel. A magnetic field is applied to a region of the channel in electrochemical contact with a working electrode to accumulate the metal particles in the region of the channel in electrochemical contact with the working electrode (steps 1a). In the absence of the molecule of interest, the analyte conjugated to the metal particle and the magnetic particle is accumulated by an applied magnetic field in a region of the channel in electrochemical contact with the working electrode (step 1a, top). In the presence of the molecule of interest, the molecule of interest and the analyte bound to the metal particle competitively bind to the antibody bound to a magnetic particle. In this case, a portion of the molecule of interest remains bound to the metal particle in the region of the channel in electrochemical contact with the working electrode, and a portion of the analyte bound to the metal particle flows downstream from the region of the channel in electrochemical contact with the working electrode (step 1a, bottom). Next the metal particles remaining in the region of the channel in electrochemical contact with the working electrode are contacted with an oxidant, and oxidized to form metal ions (step 1b). The metal ions are then electrochemically detected as described above (steps 1c and 1d) to detect and/or quantify the molecule of interest. As the concentration of the molecule of interest increases, one would expect to observe a decreased electrochemical signal at the working electrode. A control experiment can be simultaneously performed in the same channel using a metal particle-magnetic particle conjugate that does not competitively bind with the molecule of interest (steps 2a-2d). This control experiment should always provide an electrochemical signal to confirm the test was successfully performed.

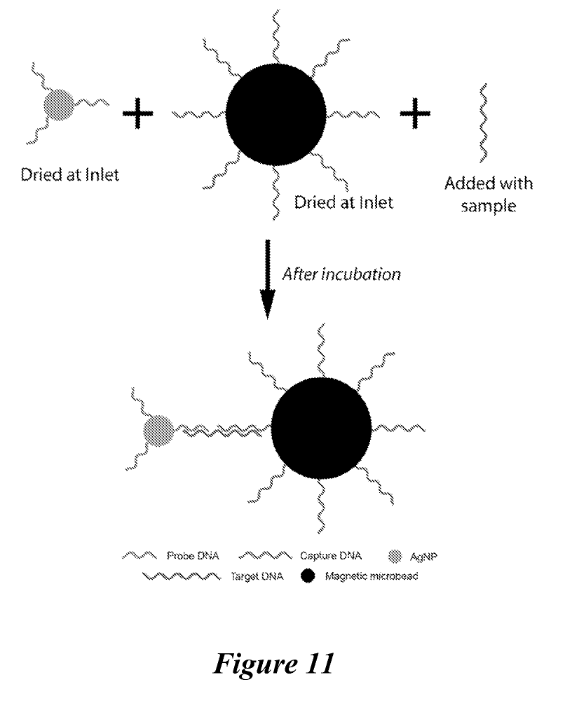

An example of a method for the electrochemical detection of a polynucleotide (e.g., DNA) is schematically illustrated in FIG. 11. In this embodiment, an analyte (e.g., a polynucleotide such as a single strand of DNA) can be contacted with a first recognition element (e.g., a first polynucleotide probe having a complementary sequence to a first portion of the analyte) and a second recognition element (e.g., a second polynucleotide probe having a complementary sequence to a second portion of the analyte). A metal particle (e.g., a silver nanoparticle) is bound to the first recognition element and a magnetic particle (e.g., a magnetic microbead) is bound to the second recognition element. The analyte can then be electrochemically detected using the method illustrated in FIG. 9. Briefly, the analyte conjugated to the metal particle and the magnetic particle can be flowed along a channel (step a), and accumulated by an applied magnetic field in a region of the channel in electrochemical contact with a working electrode. Once accumulated, the metal particles are contacted with an oxidant, and oxidized to form metal ions (step b). The metal ions can then be electrochemically detected (steps 1c and 1d) to detect and/or quantify the analyte.

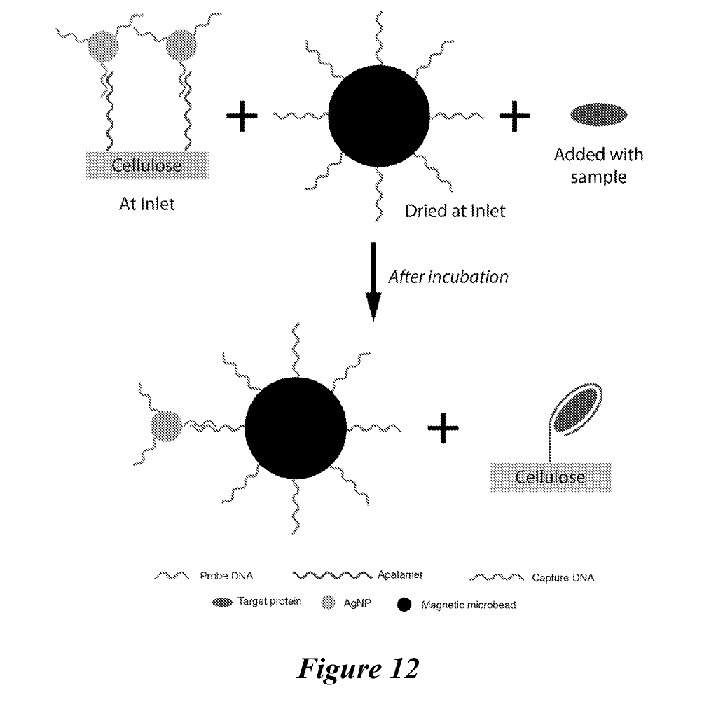

An example of a method for the electrochemical detection of a molecule of interest via a surrogate is schematically illustrated in FIG. 12. In this embodiment, a surrogate conjugated to a fixed support is used to detect a molecule of interest. A fixed analyte support (e.g., an aptamer that specifically binds the molecule of interest) is immobilized on a surface (e.g., on a porous hydrophilic substrate, such as paper, present in the fluid inlet of a device). A surrogate (e.g., a recognition element for the aptamer such as a polynucleotide probe having a complementary sequence to a portion of the aptamer) is bound to the fixed analyte support. A metal nanoparticle (e.g., a silver nanoparticle) is bound to the surrogate. The surrogate -fixed analyte support conjugate is contacted with the molecule of interest. The molecule of interest binds to the fixed analyte support, displacing the surrogate bound to a metal nanoparticle. Once displaced from the fixed analyte support, the surrogate bound to a metal nanoparticle is contacted with and binds to a recognition element for the surrogate (e.g., a polynucleotide having a complementary sequence to a portion of the surrogate). The recognition element for the surrogate is bound to a magnetic particle. The surrogate can then be electrochemically detected using the method illustrated in FIG. 9. Briefly, the surrogate conjugated to the metal particle and the magnetic particle can be flowed along a channel (step a), and accumulated by an applied magnetic field in a region of the channel in electrochemical contact with a working electrode. Once accumulated, the metal particles are contacted with an oxidant, and oxidized to form metal ions (step b). The metal ions can then be electrochemically detected (steps 1c and 1d) to detect and/or quantify the surrogate. Because the concentration of the surrogate is proportional to the concentration of the molecule of interest, the molecule of interest can be detected and/or quantified by extension.

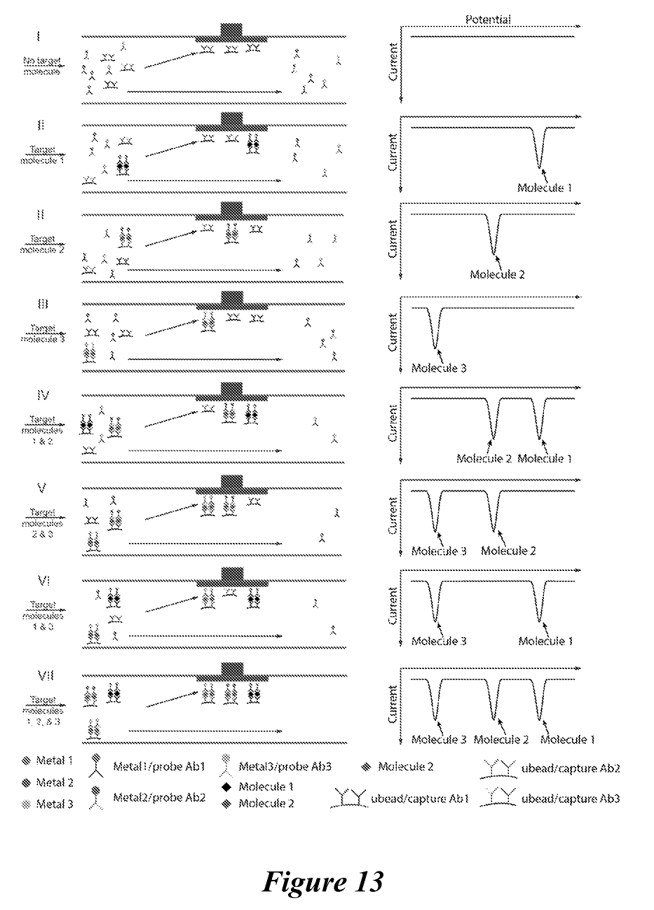

Also provided are methods for the simultaneous electrochemical detection of multiple analytes. FIG. 13 illustrates an example method for the simultaneous detection and/or quantification of multiple analytes (molecules 1-3). The first analyte (molecule 1) is bound to a first antibody and a second antibody. A first metal particle (comprising metal 1) is bound to the first antibody and a magnetic particle (e.g., a magnetic microbead) is bound to the second antibody. The second analyte (molecule 2) is bound to a third antibody and a fourth antibody. A second metal particle (comprising metal 2) is bound to the third antibody and a magnetic particle (e.g., a magnetic microbead) is bound to the fourth antibody. The third analyte (molecule 3) is bound to a fifth antibody and a sixth antibody. A third metal particle (comprising metal 3) is bound to the fifth antibody and a magnetic particle (e.g., a magnetic microbead) is bound to the sixth antibody. Metal 1, metal 2, and metal 3 are selected to be different and possess distinct reduction potentials, such that the electrochemical signals from metal 1, metal 2, and metal 3 can be individually resolved at the working electrode. For example, the metals can be selected such that they possess at least a 200 mV separation (peak-to-peak) in reduction potential, such that the metals can be individually resolved by anodic stripping voltammetry. The analytes can then be electrochemically detected using the method illustrated in FIG. 9. The analytes, each conjugated to the metal particle and the magnetic particle are flowed along a channel (step a), and accumulated by an applied magnetic field in a region of the channel in electrochemical contact with a working electrode. Once accumulated, the metal particles (metal particle 1, metal particle 2, and/or metal particle 3) are contacted with an oxidant, and oxidized to form metal ions (step b). The metal ions can then be electrochemically detected (steps 1c and 1d) to detect and/or quantify the first analyte, the second analyte, the third analyte, or combinations thereof. As illustrated in the hypothetical voltammograms for various mixtures of analytes, because metal 1, metal 2, and metal 3 are selected to be different and possess distinct reduction potentials, the electrochemical signals from metal 1, metal 2, and metal 3 can be individually resolved at the working electrode. This allows for the simultaneous detection and/or quantification of multiple analytes.

Devices

Also provided are devices for the electrochemical detection of analytes. The devices can be used to practice the electrochemical detection methods described above. The devices can comprise a channel defining a path for fluid flow from a fluid inlet to a fluid outlet, a working electrode positioned in electrochemical contact with a region of the channel, and a localization element configured to accumulate the analyte conjugated to the metal particle (i.e., the analyte conjugate) in the region of the channel in electrochemical contact with the working electrode. As described above, the localization element can be any feature that is configured to increase the concentration of the analyte conjugate in the region of the channel in electrochemical contact with the working electrode in the presence of fluid flow through the channel. For example, the localization element can be a physical barrier disposed in the region of the channel in electrochemical contact with the working electrode (e.g., a material configured to physically entrap the analyte conjugate), one or more localization electrodes configured to apply an electric field to the region of the channel in electrochemical contact with the working electrode (e.g., configured to electrophoretically localize the analyte conjugate), a magnet configured to apply a magnetic field in the region of the channel in electrochemical contact with the working electrode, or a combination thereof. Devices can further include a counter electrode, a reference electrode, or combinations thereof in electrochemical contact with the channel. Optionally, if desired for a particular detection method, the devices can further include a second working electrode positioned in electrochemical contact with a second region of the channel, and a second localization element configured to accumulate an analyte conjugated to a metal particle (i.e., an analyte conjugate) in the second region of the channel in electrochemical contact with the second working electrode.

Devices can further comprise an engageable platform that can be translocated from a retracted position to a deployed position. When the engageable platform is in the retracted position, the engageable platform is fluidly independent from the channel (e.g., engageable platform is positioned in a region of the device such that the engageable platform is not in fluid contact with the channel). When the engageable platform is in the deployed position, the engageable platform is in fluid contact with the region of the channel in electrochemical contact with the electrode (e.g., engageable platform is positioned in fluid contact with the region of the channel in electrochemical contact with the electrode). In some embodiments, when the engageable platform is in retracted position, the path for fluid flow from the fluid inlet to the fluid outlet is continuous, such that fluid can flow from the fluid inlet to the fluid outlet, and when the engageable platform is in the deployed position, the path for fluid flow from the fluid inlet to the fluid outlet is interrupted, such that fluid cannot flow from the fluid inlet to the fluid outlet.

The engageable platform can be provided, for example, as a portion of a translocatable layer of a multilayer microfluidic device, as described in more detail below. Alternatively, the engageable platform can be provided independently from one or more layers that combine to form a microfluidic device (e.g., as part of a translocatable region within a stationary layer of a multilayer microfluidic device). The engageable platform can be formed from a porous, hydrophilic material, such as paper. An oxidant can be disposed on the engageable platform (e.g., adsorbed or absorbed so the engageable platform). The oxidant can be any suitable oxidant as described above (e.g., potassium permanganate).

The devices described herein can be fabricated from any suitable material or combination of materials. In some embodiments, the devices are paper-based microfluidic devices. Paper-based microfluidic devices include a channel (i.e., a path such as a conduit, through which one or more fluids can flow) formed within a layer of a porous, cellulosic substrate. The channel can be a void space through which a fluid can flow (i.e., a hollow channel), a porous hydrophilic substrate such as paper through which fluid flows by wicking (i.e., a filled channel), or a combination thereof. The dimensions of the channel within the layer of porous, cellulosic substrate are defined by a hydrophobic boundary that substantially permeates the thickness of the porous, cellulosic substrate, so as to form a boundary for fluid flow from the channel to a region on the porous, cellulosic substrate outside of the channel, thereby directing fluid flow along the channel.

The channel can be patterned within a layer of a porous, cellulosic substrate using any suitable method known in the art. For example, the channel can be patterned by wax printing. In these methods, an inkjet printer is used to pattern a wax material on the porous, cellulosic substrate. Many types of wax-based solid ink are commercially available and are useful in such methods as the ink provides a visual indication of the location of the channels. However, it should be understood, that the wax material used to form the channels does not require an ink to be functional. Examples of wax materials that maybe used include polyethylene waxes, hydrocarbon amide waxes or ester waxes. Once the wax is patterned, the porous, cellulosic substrate is heated (e.g., by placing the substrate on a hot plate with the wax side up at a temperature of 120° C.) and cooled to room temperature. This allows the wax material to substantially permeate the thickness of the porous, cellulosic substrate, so as to form a hydrophobic boundary that defines the dimensions of the channel. At this point, the resulting channel is a filled channel, as the channel defined by the hydrophobic boundary includes a porous hydrophilic substrate (the porous, cellulosic substrate) through which fluid can flow by wicking. If desired for device design, a hollow channel can be formed by removing the porous, cellulosic substrate within the hydrophobic boundary, thereby forming a void space through which a fluid can flow.

In some embodiments, the porous, cellulosic substrate used to form the paper-based microfluidic device is flexible. For certain applications, it is preferable that the cellulosic substrate can be folded, creased, or otherwise mechanically shaped to impart structure and function to the paper-based device formed from the cellulosic substrate. Examples of suitable porous, cellulosic substrates for the fabrication of paper-based microfluidic devices include cellulose; derivatives of cellulose such as nitrocellulose or cellulose acetate; paper (e.g., filter paper, chromatography paper); woven cellulosic materials; and non-woven cellulosic materials.

In some embodiment, the porous, cellulosic substrate is paper. Paper is inexpensive, widely available, readily patterned, thin, lightweight, and can be disposed of with minimal environmental impact. Furthermore, a variety of grades of paper are available, permitting the selection of a paper substrate with the weight (i.e., grammage), thickness and/or rigidity and surface characteristics (i.e., porosity, hydrophobicity, and/or roughness), desired for the fabrication of a particular paper-based device. Suitable papers include, but are not limited to, chromatography paper, card stock, filter paper, vellum paper, printing paper, wrapping paper, ledger paper, bank paper, bond paper, blotting paper, drawing paper, fish paper, tissue paper, paper towel, wax paper, and photography paper.

In certain embodiments, the localization element can comprise a magnet configured to apply a magnetic field in the region of the channel in electrochemical contact with the working electrode. In these embodiments, the devices can comprise a channel defining a path for fluid flow from a fluid inlet to a fluid outlet, an electrode positioned in electrochemical contact with a region of the channel, and a magnet configured to apply a magnetic field to the region of the channel positioned in electrochemical contact with the electrode.

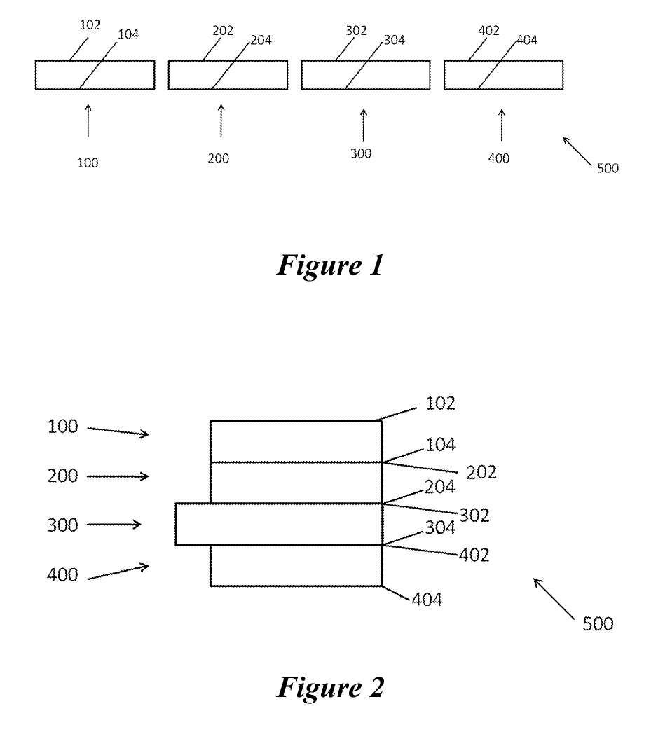

An example device employing a magnetic localization element is illustrated in FIGS. 1-8. Referring now to FIG. 1, the device (500) includes a first layer (100) having a top surface (102) and a bottom surface (104), a second layer (200) having a top surface (202) and a bottom surface (204), a third layer (300) having a top surface (302) and a bottom surface (304), and a fourth layer (400) having a top surface (402) and a bottom surface (404). Referring now to FIG. 2, when the device (500) is assembled, the bottom surface of the first layer (104) is in contact with the top surface of the second layer (202), the bottom surface of the second layer (204) is in contact with the top surface of the third layer (302), and the bottom surface of the third layer (304) is in contact with the top surface of the fourth layer (404).

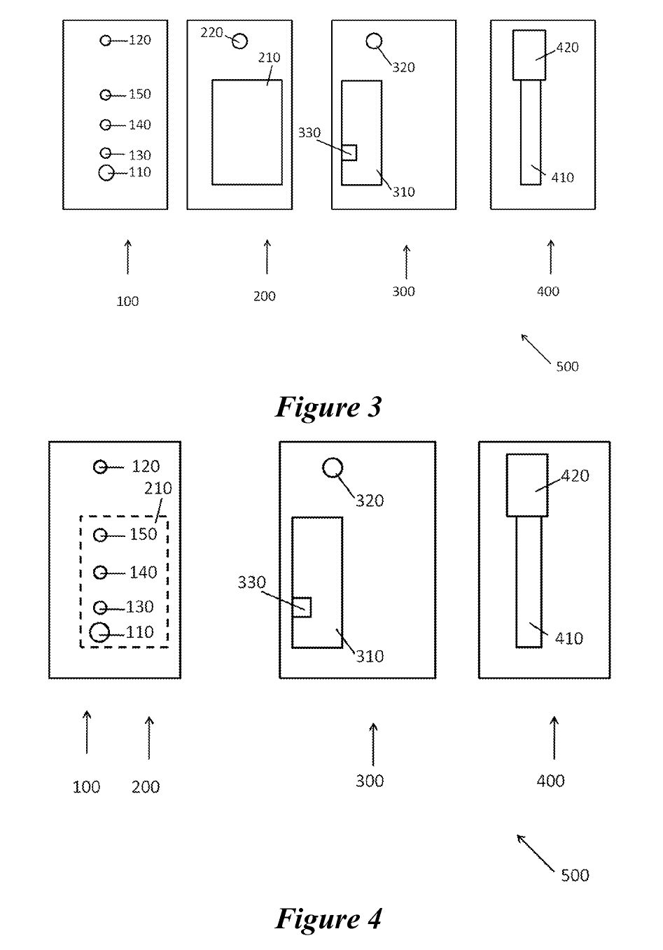

Referring now to FIG. 3, the first layer (100) includes a fluid inlet (110) defining a path for fluid flow from the top surface of the first layer (102) to the bottom surface of the first layer (104), a fluid outlet (120) defining a path for fluid flow from the bottom surface of the first layer (104) to the top surface of the first layer (102), and a working electrode (130) disposed on the bottom surface of the first layer (104). The first layer (100) can also include a reference electrode (140) disposed on the bottom surface of the first layer (104) and a counter electrode (150) disposed on the bottom surface of the first layer (104). The second layer (200) includes a hydrophobic boundary defining a channel (210) for fluid flow within the second layer, and a port (220) defining a path for fluid flow from the bottom surface of the second layer (204) to the top surface of the second layer (202). The third layer (300) includes a hydrophobic boundary defining a channel (310) for fluid flow within the third layer, a port (320) defining a path for fluid flow from the bottom surface of the third layer (304) to the top surface of the third layer (302), and an engageable platform (330) disposed within the third layer. In some embodiments, the platform (330) comprises an oxidant, as described above. The fourth layer (400) comprises a channel (410) defining a path for fluid flow within the fourth layer formed from a porous hydrophilic material, and a sink (420) fluidly connected to the channel (420) and formed from a porous hydrophilic material.

The device is assembled by aligning the four layers as shown in FIG. 2. Referring now to FIG. 4, which illustrates the first layer (100) aligned with the second layer (200), the first layer (100) is aligned with the second layer (200) such that the working electrode (130), reference electrode (140), and counter electrode (150) are in electrochemical contact with a region of the channel (210) for fluid flow within the second layer, the fluid inlet (110) is fluidly connected to the channel (210) for fluid flow within the second layer, and the fluid outlet (120) is fluidly connected to the port 220.

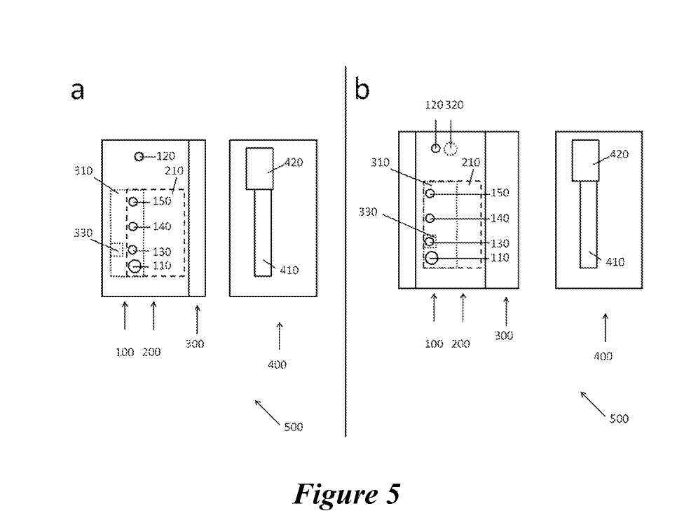

FIG. 5 illustrates the first layer (100) aligned with the second layer (200) and the third layer (300) in two different positions. In position 1 (the retracted position), shown in FIG. 5A, the first layer (100), the second layer (200), and the third layer (300) are aligned such that the fluid inlet (110), working electrode (130), reference electrode (140), counter electrode (150), and the channel (210) for fluid flow within the second layer are all aligned over the channel (310) for fluid flow within the third layer. In addition, the fluid outlet (120) is fluidly connected to port 220 and port 320, and the engageable platform (330) is fluidly independent from (i.e., not in fluid contact with) the channel (210) for fluid flow within the second layer. In position 2 (the deployed position), shown in FIG. 5B, the first layer (100), the second layer (200), and the third layer (300) are aligned such that the fluid inlet (110), working electrode (130), reference electrode (140), counter electrode (150), and the channel (210) for fluid flow within the second layer remain aligned over the channel (310) for fluid flow within the third layer. However, port 320 is no longer in fluid contact with port 220, and the engageable platform (330) is now positioned in fluid contact with the channel (210) for fluid flow within the second layer and aligned with the working electrode (130). The alignment of the layers of the device can be transitioned from position 1 to position to by translocation of the third layer (300) relative to the first layer (100) and the second layer (200).

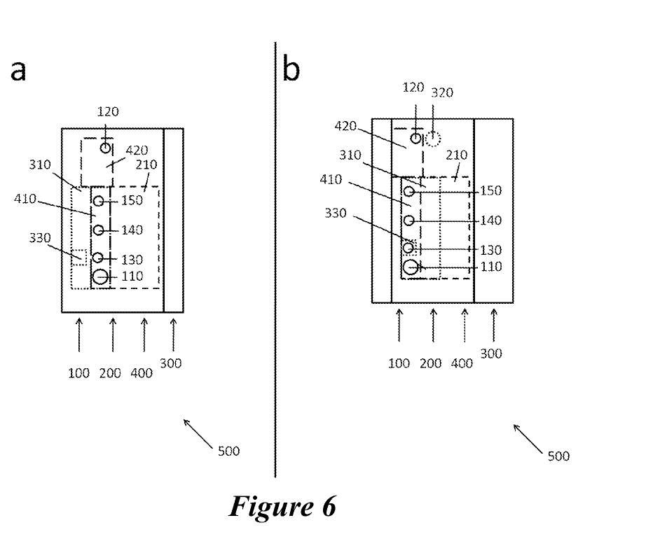

FIG. 6 illustrates a top view of the assembled device (500) with the first layer (100) aligned with the second layer (200), the third layer (300), and the fourth layer (400) in two different positions. In position 1 (the retracted position), shown in FIG. 6A, the first layer (100), the second layer (200), and the third layer (300) are aligned such that the fluid inlet (110), working electrode (130), reference electrode (140), counter electrode (150), and the channel (210) for fluid flow within the second layer are all aligned over the channel (310) for fluid flow within the third layer. The fourth layer (400) is aligned such that the channel (410) is aligned beneath the fluid inlet (110), working electrode (130), reference electrode (140), counter electrode (150), and the channel (210) for fluid flow within the second layer, and such that the channel (410) is in fluid contact with the channel (310) for fluid flow within the third layer. In addition, the fluid outlet (120), port 220, port 320, and sink (420) are aligned so as to be fluidly connected. In position 1, the first layer (100), the second layer (200), the third layer (300), and the fourth layer (400) are aligned so as to form a continuous path for fluid flow from the fluid inlet (110), to the channel (210) for fluid flow within the second layer, to the channel (310) for fluid flow within the third layer, to channel 410, to sink 420, to port 320, port 220, to the fluid outlet (120). The engageable platform (330) is fluidly independent from (i.e., not in fluid contact with) the channel (210) for fluid flow within the second layer. In position 2 (the deployed position), shown in FIG. 6B, the first layer (100), the second layer (200), and the third layer (300) are aligned such that the fluid inlet (110), working electrode (130), reference electrode (140), counter electrode (150), and the channel (210) for fluid flow within the second layer remain aligned over the channel (310) for fluid flow within the third layer. The fourth layer (400) remains aligned such that the channel (410) is aligned beneath the fluid inlet (110), working electrode (130), reference electrode (140), counter electrode (150), and the channel (210) for fluid flow within the second layer, and such that the channel (410) is in fluid contact with the channel (310) for fluid flow within the third layer. However, port 320 is no longer in fluid contact with port 220 or sink 420, such that the path for fluid flow from sink 420 to port 220 is interrupted. The engageable platform (330) is now positioned in fluid contact with the channel (210) for fluid flow within the second layer and aligned with the working electrode (130). The alignment of the layers of the device can be transitioned from position 1 to position to by translocation of the third layer (300) relative to the first layer (100), the second layer (200), and the fourth layer (400).

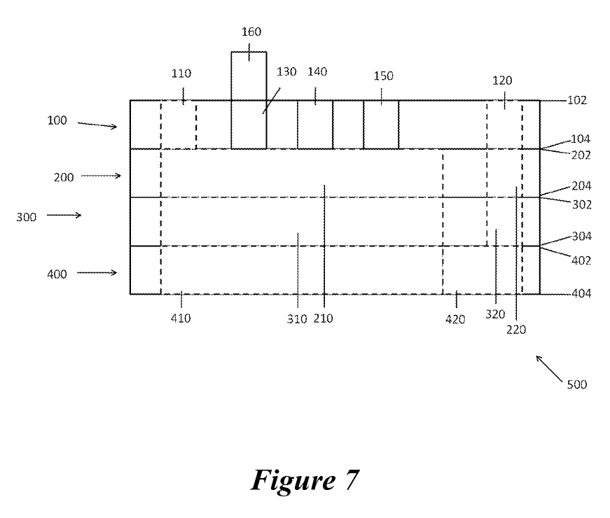

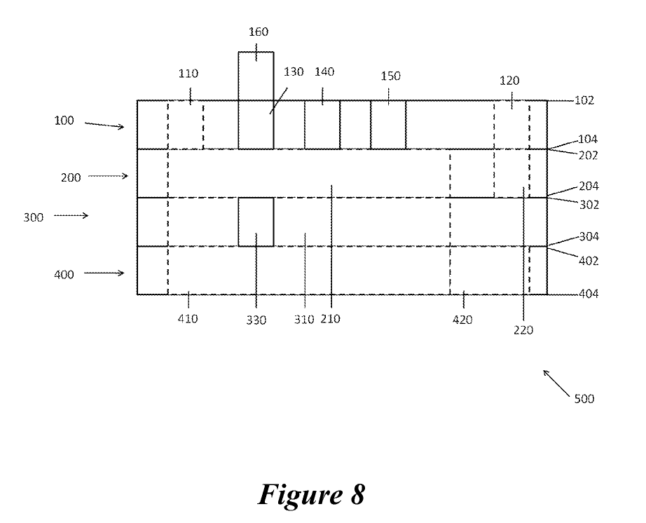

Referring now to FIGS. 7 and 8, the device (500) can further comprise a magnet (160). The magnet (160) is aligned with the working electrode (130) so as to apply a magnetic field within the region of the channel (210) in the second layer in electrochemical contact with the working electrode (130).

If desired, a reagent for the detection of a molecule of interest can be deposited at the fluid inlet. Optionally, an indicator can be disposed on the sink, the port in the third layer, the port in the second layer, or combinations thereof. The indicator can be a dye that is transported to the fluid outlet by the fluid flowing through the device, thereby indicating completion of an assay. In certain embodiments, the first layer, the second layer, and the fourth layer are fabricated from a single (integral) piece of paper that is folded to form the device.

Referring now to FIG. 27, also provided are devices for the electrochemical detection of an analyte that are configured to simultaneously perform a control assay in the same channel as the analyte detection. The example device is similar to the device illustrated in FIGS. 1-8; however, the device includes four electrodes disposed on the bottom surface of the first layer: a first working electrode (a; analyte working electrode), a second working electrode (b; control working electrode), a reference electrode (c), and a counter electrode (d). All four electrodes are positioned in electrochemical contact with a region of the channel for fluid flow within the second layer. The device also includes a first magnet aligned with the first working electrode so as to apply a magnetic field within the region of the channel in the second layer in electrochemical contact with the first working electrode, and a second magnet aligned with the second working electrode so as to apply a magnetic field within the region of the channel in the second layer in electrochemical contact with the second working electrode.

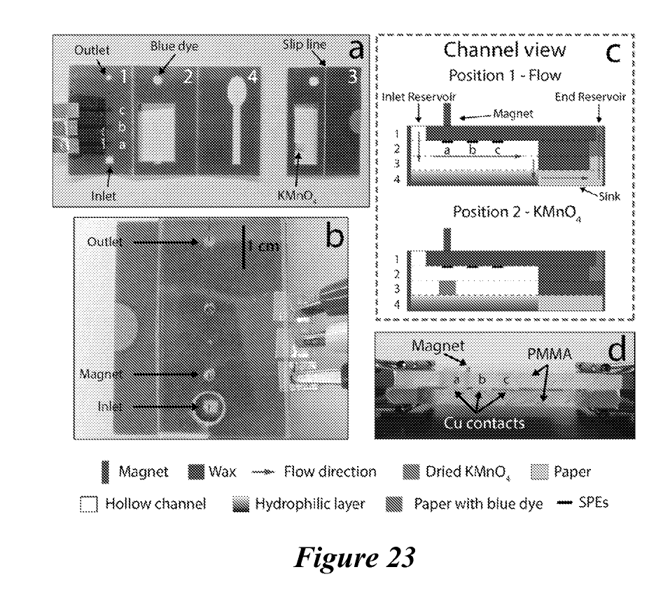

The fluid inlet comprises a porous hydrophilic substrate, such as paper, onto which reagents for the detection of the molecule of interest (e.g., an analyte bound to a metal particle and an antibody for the analyte bound to a magnetic particle in the case of the method schematically illustrated in FIG. 10) can be deposited. The fluid inlet comprises a hydrophilic barrier such that the two reagents can be isolated from one another prior to being contacted with the molecule of interest. The device also includes a control platform in fluid contact with the channel in the second layer downstream of the first working electrode but upstream of the second working electrode (illustrated as a rectangle between electrode a and b in FIG. 23). A control complex (e.g., a metal particle-magnetic particle conjugate that does not competitively bind with the molecule of interest in the case of the method schematically illustrated in FIG. 10) can be deposited on the control platform. When a fluid comprising the molecule of interest is applied to the fluid inlet, the assay for the detection of the analyte is performed, as described above, at the first working electrode. The fluid flowing through the channel also draws the control complex from the control platform. The control complex is flowed along the channel, and accumulated by an applied magnetic field in a region of the channel in electrochemical contact with the second electrode (providing for the control experiment).

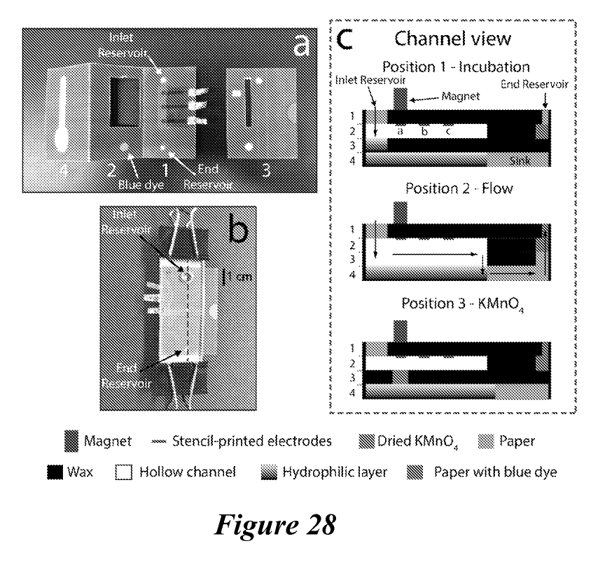

Referring now to FIG. 28, also provided are devices for the electrochemical detection of analytes that can provide for the incubation of a molecule of interest with reagents for the detection of the molecule of interest prior to initiating fluid flow through the device. The example device includes four layers as in device illustrated in FIGS. 1-8. However, the third layer of the device can be translocated between three positions. In position 1 (incubation position, panel c), a hydrophobic region of layer three fluidly isolates the channel in the second layer from the channel in the fourth layer. In this embodiment, fluid can be added to the fluid inlet; however, fluid flow through the device does not commence. The fluid inlet can comprise a porous hydrophilic substrate, such as paper, onto which reagents for the detection of the molecule of interest (which can be, for example a first antibody bound to a metal nanoparticle and a second antibody bound to a magnetic particle in the case of the method schematically illustrated in FIG. 9) can be deposited. A fluid sample comprising the molecule of interest (e.g., the analyte in the case of the method schematically illustrated in FIG. 9) can then be applied to the fluid inlet where it contacts the reagents and is incubated. Following incubation, the third layer can be translocated to position 2 (flow position, panel c) allowing fluid to flow to the fluid outlet of the device. Once flow is complete, the third layer can be translocated to position 3 (deployed position, panel c), bringing an engageable platform comprising an oxidant into fluid contact with the channel in second layer. The analyte can then be electrochemically detected using the methods described above.

If desired, the devices described herein can be affixed to or secured within a polymer, metal, glass, wood, or paper support structure to facilitate handling and use of the device. In some embodiments, the devices described herein are affixed to or secured within an inert, non-absorbent polymer such as a polyether block amide (e.g., PEBAX®, commercially available from Arkema, Colombes, France), a polyacrylate, a polymethacrylate (e.g., poly(methyl methacrylate)), a polyimide, polyurethane, polyamide (e.g., Nylon 6,6), polyvinylchloride, polyester, (HYTREL®, commercially available from DuPont, Wilmington, Del.), polyethylene (PE), polyether ether ketone (PEEK), fluoropolymers such as polytetrafluoroethylene (PTFE), perfluoroalkoxy, fluorinated ethylene propylene, or a blend or copolymer thereof. Silastic materials and siliconbased polymers can also be used.

The devices described herein can be coupled to a power supply and optionally to one or more additional suitable features including, but not limited to, a voltmeter, an ammeter, a multimeter, an ohmmeter, a signal generator, a pulse generator, an oscilloscope, a frequency counter, a potentiostat, or a capacitance meter. The devices described herein can also be coupled to a computing device that performs arithmetic and logic operations necessary to process the electrochemical signals produced by the device (e.g., to determine analyte concentration, etc.).

The devices and methods described herein are inexpensive, user friendly (they employ electrochemical detection without any washing steps), sensitive, portable, robust (they employ metal particles for signal amplification as opposed to enzymes), efficient, rapid (completion of analysis in 4.6 min), and can detect low concentrations (767 fM). As such, the device and methods are well suited for use in numerous sensing applications.

For example, the devices and methods described herein can be used in clinical and healthcare settings to detect and/or quantify biomarkers to identify risk for, diagnosis of, or progression of a pathological or physiological process in a subject. Examples of biomarkers include proteins, hormones, prohormones, lipids, carbohydrates, DNA, RNA, and combinations thereof.

The devices and methods described herein can be used in POC applications to diagnose infections in a patient (e.g., by measuring serum antibody concentrations or detect antigens). For example, the devices and methods described herein can be used to diagnose viral infections (e.g., HIV, hepatitis B, hepatitis C, rotavirus, influenza, polio, measles, yellow fever, rabies, dengue, or West Nile Virus), bacterial infections (e.g., E. coli, C. tetani, cholera, typhoid, diphtheria, tuberculosis, plague, Lyme disease, or H. pylori), and parasitic infections (e.g., toxoplasmosis, Chagas disease, or malaria). The devices and methods described herein can be used to rapidly assesses the immune status of people or animals against selected vaccine-preventable diseases (e.g. anthrax, human papillomavirus (HPV), diphtheria, hepatitis A, hepatitis B, haemophilus influenzae type b (Hib), influenza (flu), Japanese encephalitis (JE), measles, meningococcal, mumps, pertussis, pneumococcal, polio, rabies, rotavirus, rubella, shingles (herpes zoster), smallpox, tetanus, typhoid, tuberculosis (TB), varicella (chickenpox), yellow fever). The devices and methods described herein can be used to rapidly screen donated blood for evidence of viral contamination by HIV, hepatitis C, hepatitis B, and HTLV-1 and -2. The devices and methods described herein can also be used to measure hormone levels. For example, the devices and methods described herein can be used to measure levels of human chorionic gonadotropin (hCG) (as a test for pregnancy), Luteinizing Hormone (LH) (to determine the time of ovulation), or Thyroid Stimulating Hormone (TSH) (to assess thyroid function). The devices and methods described herein can be used to diagnose or monitor diabetes in a patient, for example, by measuring levels of glycosylated hemoglobin, insulin, or combinations thereof. The devices and methods described herein can be used to detect protein modifications (e.g., based on a differential charge between the native and modified protein and/or by utilizing recognition elements specific for either the native or modified protein). The devices and methods described herein can be used to administer personalized medical therapies to a subject (e.g., in a pharmacogenomic assay performed to select a therapy to be administered to a subject).

The devices and methods described herein can also be used in other commercial applications. For example, the devices and methods described herein can be used in the food and beverage industry, for example, in quality control applications or to detect potential food allergens, such as milk, peanuts, walnuts, almonds, and eggs. The devices and methods described herein can be used to detect and/or measure the levels of proteins of interest in foods, cosmetics, nutraceuticals, pharmaceuticals, and other consumer products. The devices and methods described herein can also be used to rapidly and accurately detect narcotics and biothreat agents (e.g., ricin).

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process.

Overview

Provided herein is a ready-to-use paper point-of-care platform that exploits a versatile electrochemical method for the detection and/or quantification of analytes. Specifically, it is shown that an oxidizing agent (potassium permanganate) loaded into a device can serve to spontaneously oxidize a metal nanoparticle label bound to an analyte (e.g., a silver nanoparticle (AgNP) bound to an analyte) to form metal ions in the vicinity of a working electrode. The metal ions (Ag⁺) can subsequently be electrodeposited as metal (Ag) on the working electrode. The potential can then be varied to reoxidize the deposited metal (Ag) to metal ions (AO. The intensity of the resulting voltammetric peak reflects the amount of metal deposited on the working electrode, and therefore the amount of metal nanoparticle label (and by extension analyte) present in the solution. Using this method, POC devices are provided that can detect molecules of interest at concentrations as low as 767 fM via anodic stripping voltammetry, with no washing steps or electrode modifications.

Background

There is an interest in the development of paper POC devices that are cheap, user friendly, robust, sensitive, and portable. Such devices pose an effective solution to the existing economic and healthcare accessibility problems in underdeveloped countries, as well as the growing trend in more affluent societies to become better informed in terms of its health. Although commercial paper-like sensors have been previously described (e.g., pregnancy test and glucose test strips), few paper POC devices have been successfully commercialized. Such failure to produce trustworthy paper POC devices is a combination of many factors, including poor limits of detection (LOD), high non-specific adsorption (NSA), unstable reagents, long analysis time, complex user-technology interface, detection method, and poor sensitivity. Herein, a robust and easily fabricated (no electrode modifications) paper-based platform is presented that offers a simple user-device interface (ready-to-use type of device).

Experimental

Chemicals and Materials

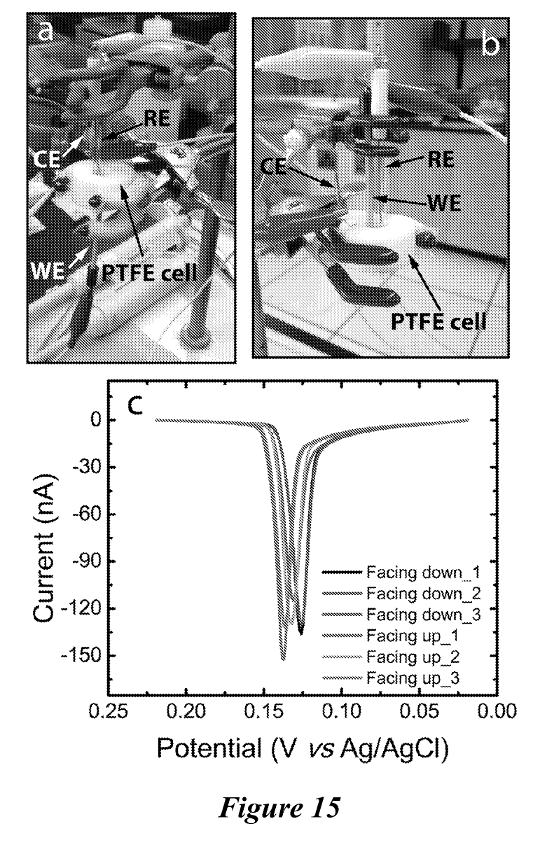

Sodium phosphate monobasic, sodium phosphate dibasic, biotin (5-fluorescein) conjugate, microtater plates (Corning 3650), and potassium permanganate (KMnO₄) were purchased from Sigma-Aldrich (St. Louis, Mo.). Sodium chloride (NaCl), sodium hydroxide (NaOH), Whatman grade 1 chromatography paper (180 μm thick, 20 cm×20 cm, linear flow rate (water) of 13 cm/30 min), microcentrifuge tubes, razor blade, two part 5-min epoxy, polytetrafluoroethylene (PTFE) tape, Parafilm paper, and Kimwipes were purchased from Fisher Scientific (Pittsburg, Pa.). AlexaFluor-647/streptavidin conjugate was purchased from Life Technologies (Grand Island, N.Y.). Streptavidin-coated magnetic microbeads (2.8 μm in diameter) were obtained from Bangs Laboratories (Fishers, Ind.). Citrate-capped silver nanoparticles (AgNP, 20.0 nm in diameter) and conductive copper tape (6.3 mm thick) were bought from Ted Pella (Redding, Calif.). Erioglaucine disodium salt (blue dye) was obtained from Acros Organics (Pittsburgh, Pa.). Conductive carbon paste (Cl-2042) was purchased from Engineered Conductive Materials (Delaware, Ohio). Neodymium cylindrical magnets ( 1/16″×½″, N48) were purchased from Apex Magnets (Petersburg, W.V.). Acrylic plates (0.6 cm thick) were obtained from Evonik Industries (Acrylite®FF). Clear nail polish was purchased from Electron Microscopy Sciences (Hatfield, Pa.). Copper epoxy (EPO-TEK 430) was acquired from Epoxy Technology (Billerica, Mass.). A4 transparency films were purchased from Office Depot. A PTFE electrochemical cell was used for all bulk-solution experiments (see FIG. 15). All bulk solution-based experiments were performed with a glassy carbon working electrode (GCE, 1.0 mm in diameter), Ag/AgCl reference electrode, and platinum wire counter electrode (CH Instruments, Austin, Tex.). Microcut polishing disks (1200 grit, 7.3 cm diameter) were purchased from Buehler (Lake Bluff, Ill.). All solutions were prepared with deionized water (<18.0 MΩ.cm, Milli-Q Gradient System, Millipore, Bedford, Mass.). Biotinylated DNA (5′d Thiol C6 SS-ACATTAAAATTC-Biotin 3′) was acquired as a powder from Biosearch Technologies (Petaluma, Calif.) and before use, it was dissolved in the appropriate volume of deionized water to yield a concentration of 100.0 μM. Phosphate buffer (1.0 M) was prepared by dissolving the appropriate amount of sodium phosphate monobasic and sodium phosphate dibasic in 0.5 L of deionized water and adjusting to the desired pH using NaOH.

Instrumentation

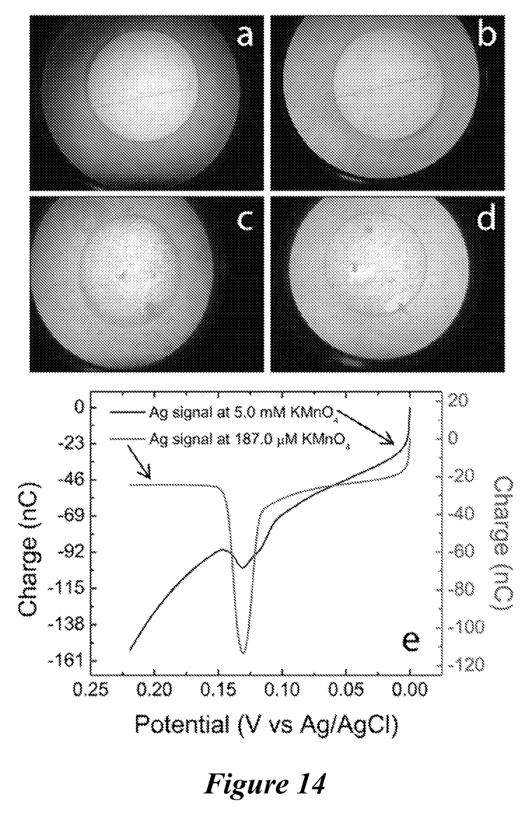

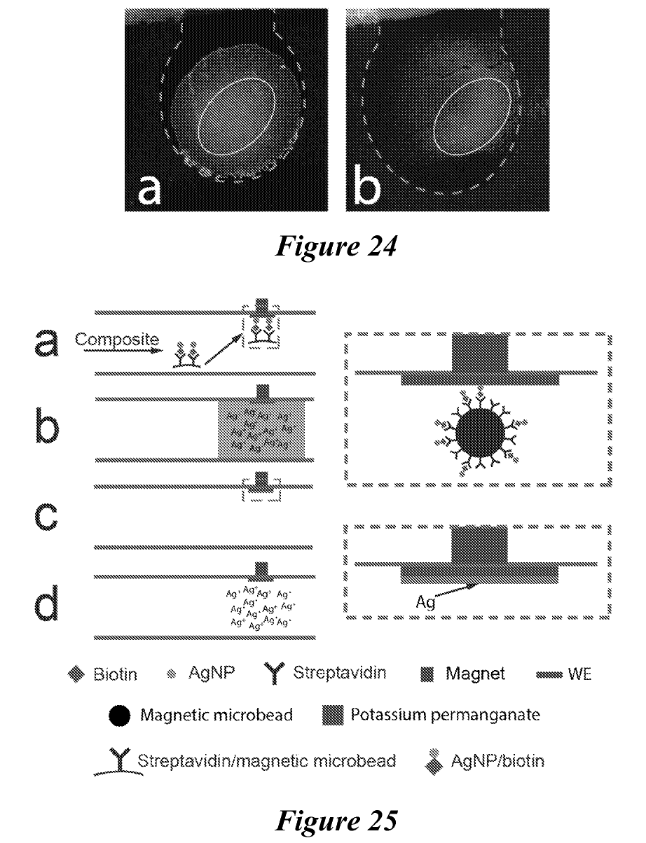

All electrochemical measurements were performed with a bipotentiostat (700E, CH Instruments, Austin, Tex.). The citrate-capped AgNP diameter reduction in the presence of KMnO₄ was characterized via Nanoparticle Tracking Analysis (NS500, NanoSight, Malvern Instruments). The microtiter plates were analyzed for fluorescence using an EnVision Multilabel reader (Perkin Elmer). UV-Vis measurements were made with a Hewlett-Packard HP8453 spectrometer. A Sorvall Legend Micro 21R Centrifuge (Thermo Scientific) was used in the synthesis of biotinylated AgNPs during the washing procedure. Mixing of all solutions was performed with a Mini Vortexer 945300 (VWR Scientific Products). Carving of the stencil was performed with an Epilog laser engraving system (Zing 16). Vacuum centrifugation was achieved with a Thermo Savant DNA120 SpeedVac Concentrator. Wax printing was obtained using a Xerox ColorQube 8570DN inkjet printer. The capture efficiency of fluorescein magnetic microbeads on the WE described below and in FIGS. 14A, 14B, 14C, 14D, 24A, and 24B were measured using a Nikon AZ100 (Nikon Co., Tokyo, Japan) microscope equipped with a mercury lamp (Nikon) and a CCD camera (Cascade, Photometrics Ltd., Tucson, Ariz.). Fluorescence images were captured using and HQ:F filter (λex=460-500 nm, λex=510-560 nm). All images were captured using 1×1 binning with 512×290 pixels and a 1 s exposure time and processed using ImageJ 1.45 s software.

oSlip Fabrication

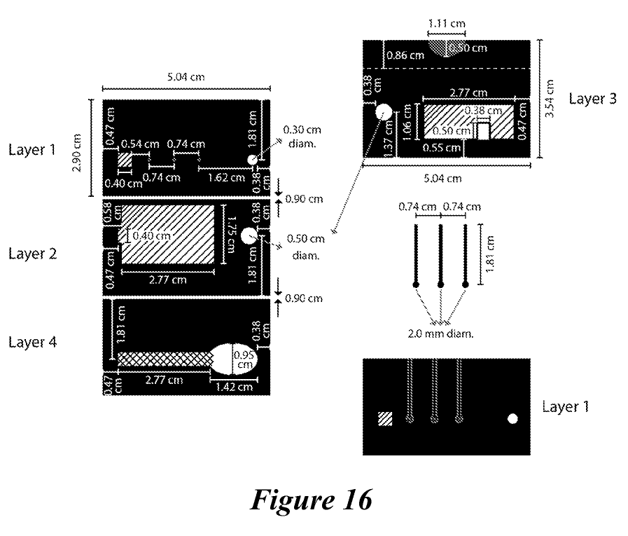

The oSlip patterns (FIG. 16, left and top right) were designed using Adobe Illustrator CS6 (version 16.0.0) and printed on Whatman grade 1 chromatography paper using a Xerox ColorQube 8570DN inkjet printer that deposited wax-based solid ink (Carrilho et al. Anal. Chem. 2009, 81, 7091-7095; Lu et al. Electrophoresis, 2009, 30, 1497-1500). Next, the paper sheet was placed in the oven at 130° C. for 50 s to melt the wax and form three-dimensional hydrophobic walls (solid black section in FIG. 16, left and top right). Note that the hydrophilic layer of Layer 4 (cross-hatched section in FIG. 16, left) was constructed by applying 60% yellow wax (Renault et al. Anal. Chem. 2013, 85, 7976-7979). After the paper was removed from the oven and cooled to 25° C., each individual device was cut from the paper sheet using scissors. The regions illustrated by the diagonal hatching (FIG. 16 left and top right) were then removed using a razor blade.

Before depositing the carbon electrodes on the device, the carbon paste was thickened by adding a 1.0 cm thick layer of carbon paste on a glass vial and placing it in an oven at 65° C. for 30 min. Next, the paste was removed from the oven, mixed with a glass rod, and re-heated in the oven at 65° C. for 5 min. The re-heating step was performed a total of two times. The thickened paste was left to cool at 25° C. until used. In addition, the stencil was designed (FIG. 16, middle right) using Adobe Illustrator CS6 (version 16.0.0) and printed on transparency films using a laser engraving system (Epilog, Model Zing 16). The finalized stencil was aligned with Layer 1 as shown in FIG. 16, bottom right and the thickened carbon paste was spread on top of the stencil using a scraper. A total of 20 devices could be stencil printed at the same time. The stencil printed carbon paste was then left to dry at 25° C. for 1 h. Next, a piece of Parafilm paper was placed on top of the circular section of the electrodes (FIG. 16, middle right) and copper epoxy was applied at the end of each electrode, immediately followed by the placement of a strip of copper tape on top of the copper epoxy. The Parafilm was removed and the devices were placed in the oven at 60° C. for 1 h to cure the copper epoxy and enhance the electronic properties of the carbon paste. Once the devices were removed from the oven, a thin layer of 5 min epoxy was placed covering all the electrodes except the circular areas. After the epoxy was completely dry (30 min at 25° C.), a thin layer of nail polish was applied over it to make the surface more hydrophobic. The nail polish was dried for 30 min at 25° C.

Modification of Citrate-Capped AgNPs with Biotin/DNA.

The citrate-capped AgNPs were biotinylated following a protocol by Alivisatos and co-workers (Sonnichsen et al. Nat. Biotech. 2005, 23, 741-745). Briefly, 10.0 μL of 100.0 μM biotin/DNA and 600.0 μL of 0.75 nM citrate-capped AgNPs were incubated at 25° C. while vortexing (level 3) for 24 h. Then, the solution's salt concentration was slowly increased to 70.0 mM NaCl and 7.0 mM phosphate buffer by adding one aliquot of a solution containing 2.5 μL of 5.0 M NaCl and 25.0 μL of 50.0 mM phosphate buffer (pH 7.0) every day for 4 days. Next, the solution's volume was slowly reduced to 250.0 μL at 40° C. using vacuum centrifugation for 3 hours. The resulting solution was centrifuged at 16,000 g for 20 min and the supernatant was removed. The silver nanoparticles were washed by re-suspending them in 600.0 μL of a solution containing 100.0 mM NaCl and 10.0 mM phosphate buffer (pH 7.0), centrifuging at 16,000 g for 20 min, and discarding the supernatant. This last washing procedure was repeated a total of three times. In order to confirm the biotinylation of the AgNPs, an aqueous solution of AlexaFluor-647/streptavidin conjugate (50.0 μg/mL final concentration) was incubated with the resulting AgNP solution at 25° C. for 30 minutes and then washed three times by re-suspending them in 600.0 μL of a solution containing 100.0 mM NaCl and 10.0 mM phosphate buffer (pH 7.0), centrifuging at 16,000 g for 20 min, and discarding the supernatant. At the same time, a control experiment was performed where 600.0 μL of 0.75 nM citrate-capped AgNPs were subjected to the same protocol described above but in the absence of biotin/DNA and finally incubated with AlexaFluor-647/streptavidin conjugate. Then, aliquots of each experiment (test and control) were placed in a microtiter plate and their fluorescence was read using a plate reader (λₑₓ=652 nm, λₑₘ=688 nm). The fluorescence recorded for the test experiment was 87% higher than that of the control experiment, confirming the biotinylation of the AgNPs.

Preparation of AgNP/Biotin/Streptavidin/Magnetic Microbeads Composite

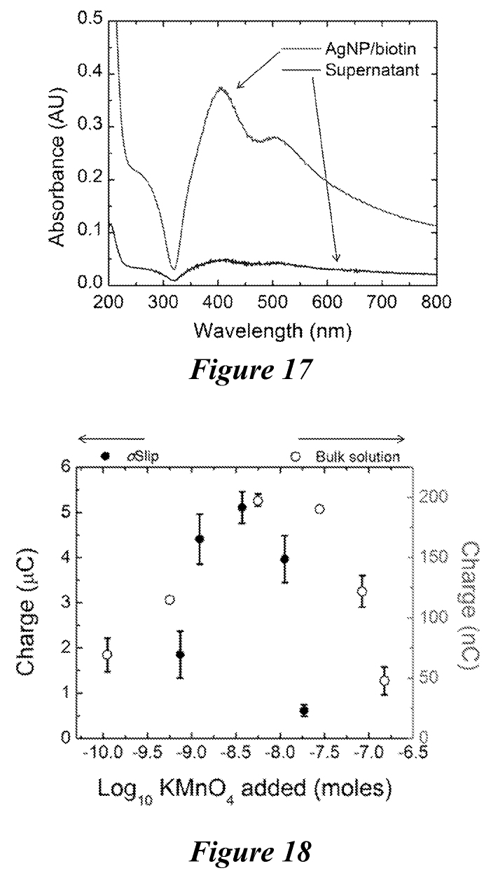

100.0 μL of stock streptavidin-coated magnetic microbeads (1.11 pM, 2.8 μm in diameter) were placed in a microcentrifuge tube and a magnet was held close to the tube for 30 s, followed by the removal of the supernatant. The microbeads were washed three times with 50.0 μL of 10.0 mM phosphate buffer (pH 7.4, PB) by placing the magnet close to the tube for 30 s and removing the supernatant between washes. After the third wash the magnetic beads were re-suspended in 200.0 μL of the previously synthesized biotinylated AgNPs solution. The AgNPs and magnetic microbeads were incubated for 30 min at 25° C. while vortexing (level 3) and then washed three times with 100.0 μL of 10.0 mM PB containing 100.0 mM NaCl by placing the magnet close to the tube for 30 s and removing the supernatant between washes. The composite formation (AgNP/biotin/streptavidin/magnetic microbead) was confirmed using UV-Vis spectroscopy (FIG. 17) at 420 nm. The trace labeled “AgNP/biotin” shows the absorbance corresponding to the solution of biotinylated AgNPs before it was incubated with the streptavidin-coated magnetic microbeads solution. The trace labeled “Supernatant” corresponds to the supernatant absorbance after incubation. The decrease in absorbance intensity at 420 nm between the traces confirms the composite formation.

Modification of Streptavidin-Coated Magnetic Microbeads with Biotin/Fluorescein Conjugate

18.0 μL of stock streptavidin-coated magnetic microbeads were placed in a microcentrifuge tube and washed three times with 100.0 μL of 10.0 mM PB by placing a magnet close to the tube for 30 s. After the third wash, the microbeads were re-suspended in an aqueous solution containing 100.0 μL of 62.0 μM biotin/fluorescein conjugate and incubated at 25° C. under vortexing (level 3) for 30 min. Next, the resulting fluorescein/magnetic microbeads were washed three times with 100.0 μL of 10.0 mM PB by placing a magnet close to the tube for 30 s. After the last wash, the beads were re-suspended in 100.0 μL of 10.0 mM PB and stored at 4° C. in the dark until used.

Optimization of the Electrochemical Signal

The maximum charge obtained in both the oSlip and bulk solution-based experiments was optimized by changing the number of moles of KMnO₄ added (while keeping the same number of moles of Ag) and then measuring the resulting charge. For the oSlip experiment, different amounts of KMnO₄ moles were dried by nitrogen flow on the square reservoir of Layer 3 (FIG. 23). After the device was assembled, 3.0 μL of stock composite (containing 908.4 pM AgNP) were added to the Inlet, immediately followed by 47.0 μL of 100.0 mM PB containing 100.0 mM NaCl. Once the Outlet turned blue, Layer 3 was slipped into position 2. 12 s after slipping the device into position 2, the WE was held at −0.6 V vs a carbon paste quasi-reference electrode (cpQRE) for 200 s. After a quiet time of 10.0 s, the potential was swept from Eᵢ=−0.5 V vs cpQRE to E_{f}=0 V vs cpQRE at ν=10 mV/s. For the bulk solution-based experiments, 125.0 μL of PB containing 100.0 mM NaCl and 50.0 μL of 75.0 pM citrate-capped AgNPs were introduced to the PTFE cell in the Facing Up setup. Then, 50.0 μL of different concentrations of KMnO₄ were added and, after 30 s, the GCE was held at −0.3 V for 200 s. Following a quiet time of 10.0 s, the electrode was swept from Eᵢ=0 V to E_{f}=0.22 V at v=10 mV/s. FIG. 18 shows that a maximum charge is obtained at 3.7 and 5.6 nmoles of KMnO4 for both the oSlip and bulk solution-based experiments, respectively. Therefore, all oSlip and bulk-solution-based experiments were performed at the mentioned amount of KMnO₄ moles. To the left of this maximum point, the charge decreases due to a lack of available KMnO₄ moles available to oxidize the AgNPs. To the right of the maximum point, the charge decreases as well. We attribute this effect to the electrical insulation of the electrode surface with MnO₂. At neutral pH, KMnO₄ is reduced to MnO₂ according to the following equation:

MnO₄⁻+2H₂O+3e [symbol] MnO₂+4OH⁻

Therefore, the higher the KMnO₄ concentration used, the more MnO₂ is produced and the more the electrode is electronically isolated. FIG. 14 shows micrograph images of a GCE before and after the electrodeposition and stripping steps (in the absence of AgNPs) at KMnO₄ concentrations of 5.0 mM and 187.0 μM. As it can be observed in the images, a thicker layer of insulator is observed at 5.0 mM. This is evidenced by a more resistive stripping wave when using 5.0 mM KMnO₄ (FIG. 14E). This effect is consistent in both the Facing Up and Facing Down setups (data not shown) as the charges measured are the same (Facing Up=214±9 nC, Facing Down 211±7 nC). (FIG. 15C).

Calibration Curve in Bulk Solution and Characterization of the Composite's AgNP Content

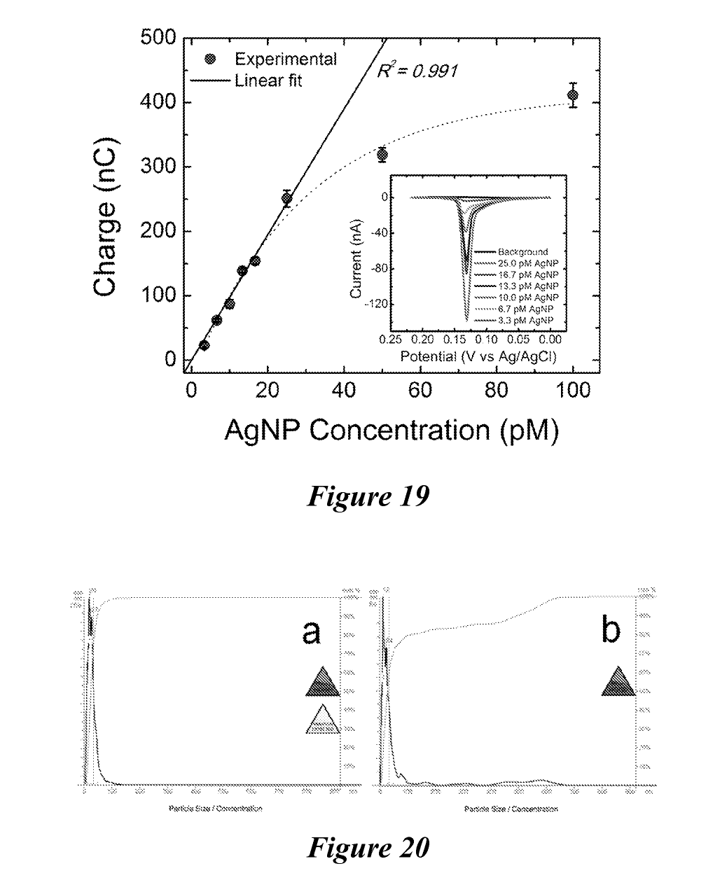

Before implementing the proposed electrochemical method (using KMnO₄) on paper, we first studied it in bulk solution. In this case, a glassy carbon working electrode (GCE, 1.0 mm in diameter), Ag/AgCl reference electrode, and platinum wire counter electrode were used in the Facing Up setup (FIG. 15). The GCE was placed tightly in its position using PTFE tape (to avoid leaking). Then, 125.0 μL of 100.0 mM PB containing 100.0 mM NaCl and 50.0 μL of 187.0 μM KMnO₄ were added to a PTFE cell. Next, 50.0 μL of an aqueous solution containing different concentrations of citrate-capped AgNPs were added to the cell and mixed. After 30 s, the GCE was held at −0.3 V for 200 s to electrodeposit Ag onto the GCE. After a quiet time of 10.0 s at 0 V, the potential was swept from 0 to 0.2 V at v=10 mV/s to oxidize Ag into Ag⁻ and obtain an anodic current transient (FIG. 19, inset). As mentioned in the main text, the charge under each stripping peak can be measured by integrating the area under the peak and dividing the obtained number by the scan rate. The calibration curve shown in FIG. 19 demonstrates there is a linear correlation (R²=0.991) between the charge measured under the stripping peaks and the concentration of AgNP added. Note that a background run in the presence of 125.0 μL of 100.0 mM PB containing 100.0 mM NaCl, 50.0 μL of 187.0 μM KMnO₄, and 50.0 μL of deionized water (no AgNP) was always performed between measurements to assure the GCE's surface was clean. After each background run, the GCE was polished with microcut paper for 1 min, rinsed with deionized water, and dried with Kimwipes (these last three steps were repeated when necessary until the GCE was free of Ag).

The concentration of AgNPs present in the composite was calculated by adding 125.0 μL of 100.0 mM PB containing 100.0 mM NaCl, 50.0 μL of 187.0 μM KMnO₄, 3.0 μL of composite, and 47.0 μL of deionized water to the PTFE cell while in the Facing Up electrochemical setup (FIG. 15). After performing the electrodeposition and stripping steps as described above, the average charge (three repetitions) measured was correlated to the concentration of AgNPs in the stock composite solution using the bulk solution-based calibration curve linear fit equation (y=1.035*10⁻⁷×−8.742*10⁻⁸).

Spontaneous Oxidation of AgNPs by KMnO₄

Two experiments were performed in order to confirm the spontaneous oxidation of AgNPs in the presence of KMnO₄. In the first one (the test), 625.0 μL of 100.0 mM PB containing 100.0 mM NaCl and 250.0 μL of 75.0 pM citrate-capped AgNPs were placed in a microcentrifuge tube. Next, 250.0 μL of 187.0 μM KMnO₄ was added and, after 30 s, the tube was placed in the nanoparticle tracking instrument (NS500, Nanosight) for analysis. In the second experiment (the control), 625.0 μL of 100.0 mM PB containing 100.0 mM NaCl, 250.0 μL of 75.0 pM citrate-capped AgNPs, and 250.0 μL of deionized water were placed in a microcentrifuge tube and analyzed. For each experiment, the solution was evaluated a total of three times. Citrate-capped AgNP diameters of 13±3 and 21±1 nm were obtained for the experiments with and without KMnO₄, respectively (one example of each experiment is provided in FIG. 20).

Optimization of KMnO₄ Resolvation Time in the oSlip

The waiting time between slipping Layer 3 into position 2 and the initiation of the electrodeposition step was optimized in order to maximize the amount of KMnO₄ that reaches the electrode surface for the oxidation of the AgNPs. This was done by drying 4.0 μL of 934.0 μM KMnO₄ on the engageable platform of Layer 3 (see FIG. 23) by nitrogen flow, assembling the oSlip, and adding 50.0 μL of 100.0 mM PB containing 100.0 mM NaCl to the fluid inlet (Inlet). Once the fluid outlet (Outlet) turned blue, Layer 3 was slipped into position 2 and after different waiting times (5, 12, 30, and 120 s), a cyclic voltammogram was recorded (from Eᵢ=0.6 V to E_{f}=−1.0 V vs cpQRE at v=100 mV/s).

FIG. 22 shows plot of peak cathodic current as a function of the waiting time. It can be observed that KMnO₄ reaches the electrode surface in 5.0 s and that waiting for longer than 12 s causes a great portion of the KMnO₄ re-solvated to diffuse away from the electrode. Note that the Ohmic drop for every oSlip was compensated before each measurement. This measurement takes a total of 10 s; therefore, the 5 s data point was obtained by fixing the potentiostat to compensate for an Ohmic drop of 4.0 kΩ. Thus, due to accuracy reasons, 12 s was chosen as the ideal waiting time for all oSlip experiments. The inset of FIG. 22 shows cyclic voltammograms of three different oSlips for the reduction of KMnO₄ after a waiting time of 12 s.

Effect of NaCl in the Electrochemical Signal

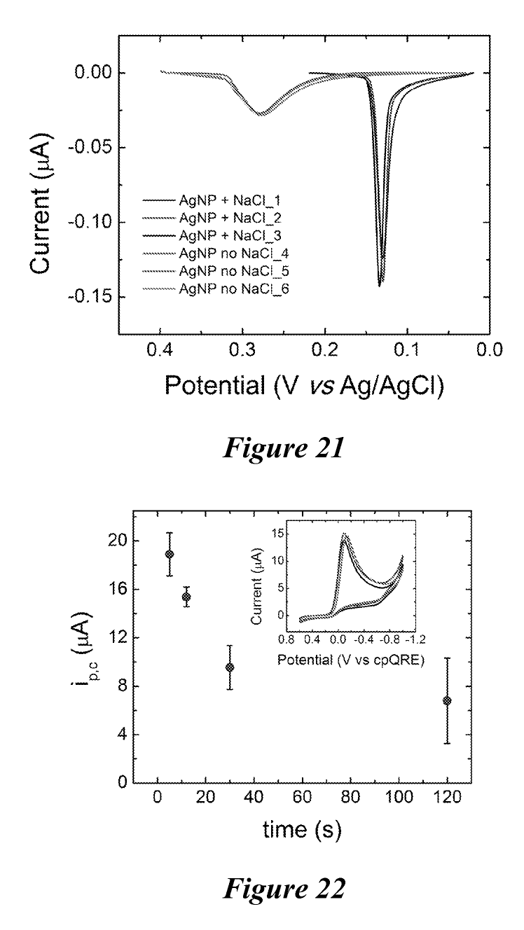

The effect of NaCl on the electrochemical signal was demonstrated by performing two bulk solution-based experiments. In the first experiment, 50.0 μL of 75.0 pM AgNP, 125.0 μL of 100.0 mM PB containing 100.0 mM NaCl, and 50.0 μL of 187.0 μM KMnO₄ were added to the PTFE cell in the facing up setup (FIG. 15A). After electrodepositing for 200 s at −0.3 V, quiet time at 0 V for 10 s, and stripping from Eᵢ=0 V E_{f}=0.22 V vs Ag/AgCl at v=10 mV/s the charge under each peak was measured. In the second experiment, 50.0 μL of 75.0 pM AgNP, 125.0 μL of 100.0 mM PB, and 50.0 μL of 187.0 μM KMnO₄ were added to the PTFE cell in the facing up setup (FIG. 15A). After electrodepositing for 200 s at −0.3 V, quiet time at 0 V for 10 s, and stripping from Eᵢ=0 V E_{f}=0.4 V vs Ag/AgCl at v=10 mV/s the charge under each peak was measured. Charges of 187±18 and 200±29 nC were recorded for the first and second experiments, respectively (FIG. 21). Although the stripping peak shape and position vary between the two experiments (Ag⁺+e [symbol] Ag E°=0.7991 V vs NHE; AgCl+e [symbol] Ag⁺+Cl⁻E°=0.2223 V vs NHE), the charges measured are not statistically different. This result can be attributed to the presence of KMnO₄ keeping Ag⁺ in solution even in the presence of high Cl⁻ concentrations that should precipitate Ag⁺ as AgCl. The experimental setups (facing up vs. facing down, FIGS. 15B and 15C) also confirm this result, as there is no statistical difference between the charges recovered (facing up=214±9 nC, facing down=211±7 nC). These numbers should be much different from each other if AgCl were to precipitate out from solution. Therefore, it can be conclude that the presence of NaCl at high concentrations does not affect the results of the proposed electrochemical detection method.

Results and Discussion

Herein, a robust and easily fabricated (no electrode modifications) paper-based platform is presented that offers a simple user-device interface (ready-to-use type of device). The detection method consists of the signal amplification of AgNP labels via their spontaneous oxidation by KMnO₄ and the subsequent electrodeposition of Ag⁺ onto the device's working electrode (WE). This deposited Ag can be later stripped off to obtain an anodic current transient that is directly proportional to the concentration of AgNP labels present. KMnO₄ is a well-known oxidizing agent (E°=1.70 V vs. NHE at neutral pH) used in many important organic and inorganic redox reactions and water disinfection. KMnO₄ was selected, among other strong oxidizing agents, due to its stability under the conditions needed for this invention.

The proposed paper platform, called oSlip, is illustrated in FIG. 23 (the dimensions are provided in FIG. 16). The oSlip is composed of 4 layers, with three of these comprising an oPAD device (Layers 1, 2, and 4) and one additional layer (Layer 3) that slips between Layers 2 and 4. The layers are numbered from top to bottom in FIG. 23A, following the order of liquid flow once the device is folded (FIG. 23B and FIG. 23D). FIG. 23C shows a cross-section of the oSlip sensor along the length of the channel (dotted line in FIG. 23B). Layer 1 has two reservoirs (FIG. 23C). One of these reservoirs, called the fluid inlet (or Inlet), had its cellulose content removed. The other reservoir, called the fluid outlet (or outlet), contains cellulose. In addition, stencil-printed carbon electrodes (rectangles labeled a, b, and c in FIG. 23C) are fabricated on the lower face of this layer (face in contact with Layer 2). Electrodes a, b, and c refer to the working electrode (WE), reference electrode (RE), and counter electrode (CE), respectively. Layer 2 contains a hollow channel and a paper reservoir (a port) loaded with a blue dye. Layer 3 is the slip layer, and it consists of a hollow channel and two paper reservoirs, one circular (a port) and the other square (an engageable platform). Finally, Layer 4 contains a hydrophilic layer (a channel) and a sink.

Prior to the initiation of the experiment, biotin-modified citrate-capped AgNP (20.0 nm in diameter) and commercially available streptavidin-coated magnetic microbeads (2.8 μm in diameter) are incubated to obtain a composite of the form AgNP/biotin/streptavidin/magnetic microbead. The AgNPs concentration present in the stock composite solution was calculated to be 533.4 pM from a bulk solution-based calibration curve of charge as a function of citrate-capped AgNP concentration (FIG. 19). In addition, 3.0 μL of blue dye is dried at 25° C. on the paper reservoir present (port) on Layer 2 and 4.0 μL of an aqueous solution of 934.0 μM KMnO₄ is dried by nitrogen flow onto the square reservoir (engageable platform) of Layer 3 (FIG. 25A and FIG. 25C). The moles of KMnO₄ needed to drive the best electrochemical signal was optimized (FIG. 18). Then, the device is assembled by folding Layers 1, 2, and 4 and placing Layer 3 between Layers 2 and 4 (FIG. 23C, position 1). Next, the assembled device is placed between two acrylic plates, compressed with paper binder clips, and a small magnet is inserted into a close-fitting hole made on the top acrylic plate which aligns it with the WE (FIG. 23D).

Once the reagents are loaded to their respective reservoirs and the device is assembled and placed in the holder, 50.0 μL of 100.0 mM phosphate buffer (PB) at pH 7.4 containing 100.0 mM NaCl and different concentrations of composite were introduced to the fluid inlet (Inlet) while Layer 3 is in position 1 (FIG. 23C). While the device is configured in position 1, the sample flows horizontally across the hollow channel (aided by the hydrophilic layer (channel) in Layer 4) and the composite present in the sample is concentrated at the WE by the magnetic field applied by the magnet positioned above the WE (FIG. 25, also see top right inset). As the composite is captured close to the WE, the remaining sample flows further through the channel and into the sink. When the sink becomes full, upward flow is initiated through the paper reservoir (port) at the right edge of Layer 3 (FIG. 23C). This upward flow picks up the blue dye pre-loaded on Layer 2 and the end of flow is signaled when the fluid outlet (Outlet) turns blue (15±2 s).

Two experiments were performed to calculate the capture efficiency of the composite at the WE. In the first experiment (Control), 2.0 μL of 10.0 mM PB containing 0.2 pM fluorescein-labeled magnetic microbeads (2.8 μm in diameter) were placed on the WE of an unassembled device and left to dry at room temperature. Once the solution was dry, its fluorescence was measured. In the second experiment (Test), 50.0 μL of 100.0 mM PB containing 100.0 mM NaCl and the same amount of fluorescein-magnetic microbead moles as in the Control experiment were added to the Inlet of an assembled device. After flow stopped (indicated by the fluid outlet turning blue), the device was left at 25° C. for 2 h, until the sample was dry. At this point, the device was opened and the fluorescence at the WE was measured. The fluorescence intensity observed at the Control WE (FIG. 24A) was 64% higher than that observed at the Test WE (FIG. 24B). Thus, the capture efficiency of the composite at the WE was determined to be 36±10%.

Once flow through the device is halted, the blue color at the fluid inlet indicates that Layer 3 needs to be pulled into position 2 (FIG. 23C) by aligning the slip line (FIG. 23A) perpendicularly to the edge of the holder. This results in placement of the square reservoir (engageable platform) containing KMnO₄ on Layer 3 directly beneath the WE, introducing the oxidizing agent to the channel. The instantaneous oxidation of AgNP into Ag⁺ by KMnO₄ is evidenced by a decrease in the AgNPs diameter from 21±1 nm to 13±3 nm (FIG. 20). As mentioned before, at this stage in the experiment there is no flow because the device is full; thus, the Ag⁻ ions produced by the presence of KMnO₄ remain close to the WE (FIG. 25B). Any loss of Ag⁺ away from the WE area is purely due to diffusion. Twelve seconds after slipping the device into position 2 (FIG. 22), the WE is held at E=−0.6 V vs. carbon-paste QRE (cpQRE) for t=200 s to electrodeposit Ag⁺ onto the WE (Ag⁺+e [symbol] Ag, FIG. 25C, also see lower right inset). Finally, after a quiet time of 10 s at −0.5 V vs cpQRE, the potential at the WE is swept from Eᵢ=−0.5 V to E_{f}=0 V (vs. cpQRE) at v=10 mV/s to obtain an anodic current transient corresponding to the oxidation of Ag into Ag⁺ (FIG. 25D).

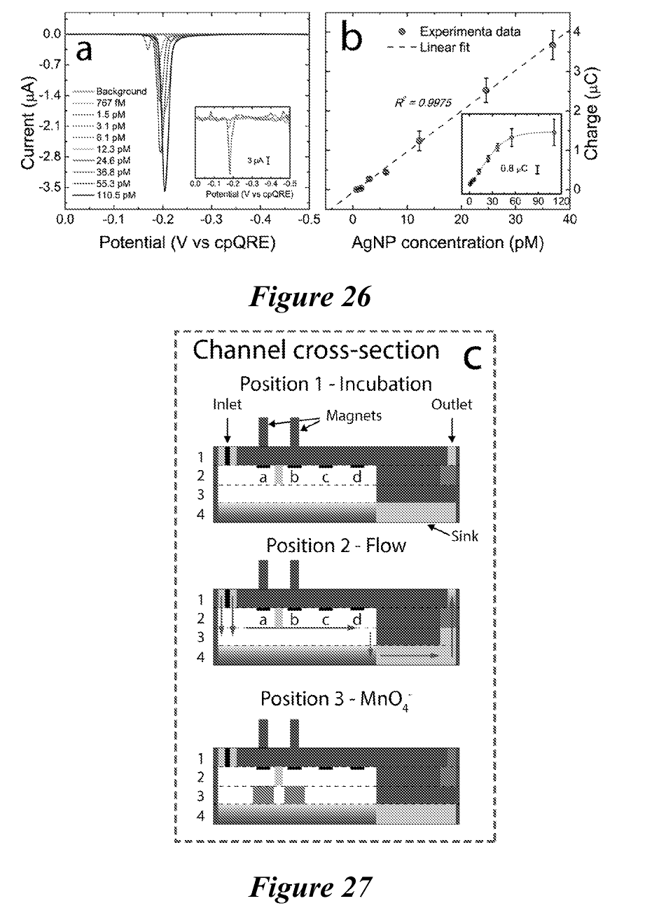

FIG. 26A shows the stripping peaks of different composite concentrations corresponding to the oxidation of Ag into Ag⁺. By integrating the area under each peak and then dividing the obtained value by the scan rate, each stripping wave could be correlated to the charge passed. FIG. 26B demonstrates there is a linear dependency (R²=0.9975) between the absolute charge measured and the concentration of AgNPs (contained in the composite) present in the sample. This proof-of-concept experiment demonstrates that both the device platform and electrochemical method function to allow for electrochemical detection and/or quantification. The electrochemical response obtained showed good sensitivity and was able to achieve the detection of AgNP concentrations as low as 767 fM (see inset of FIG. 26A). In addition, charge collection efficiency and linear range were calculated to be 13±2%, and 32.9 pM respectively. It should be noted that because in this experiment we used the same amount of KMnO₄ for each data point, while changing the concentration of AgNPs, the signal begins to plateau at 55.3 pM (FIG. 26B, inset) due to a deficiency of KMnO₄ moles available to oxidize the AgNPs. When performing the same experiment in the absence of KMnO₄, no anodic stripping signal was observed, even at AgNP concentrations where signal saturation was observed in the presence of KMnO₄ (e.g., 55.3 pM). In the absence of KMnO₄ there is a lack of electrical contact between Ag and the electrode surface (FIG. 25, top right inset). The proposed KMnO₄ method has the advantage of bringing the signal source (Ag) in close proximity to the WE (FIG. 25, bottom right inset) so that signal amplification is obtained.

The conditions under which the proof-of-concept experiment was carried out (neutral pH and salt concentration of 100.0 mM) mirror those present in human urine, which is the potential sample matrix for a wide variety of bioassays. The Kₛₚ of Ag⁺ in the presence of 100.0 mM NaCl is 1.8×10⁻⁹ M; however, no experimental evidence was found showing the precipitation of Ag⁺ as AgCl (see FIG. 21). This can be attributed to the presence of KMnO₄, keeping Ag⁺ in solution even in the presence of high concentrations of Cl⁻ ions. The pre-prototype device-to-device signal relative standard deviation (RSD, defined as the standard deviation divided by the mean signal intensity) is 13%. This value was obtained from the average RSD of different data points in the calibration curve linear range and can be considered to be an acceptable signal variation given that these devices and holders were made manually. Moreover, the signal output was obtained in only 4.6 min. It is important to mention that although electrodepositing for longer times (e.g., t=500 s) could result in detection of lower concentrations, it would also result in longer analysis times (>4.6 min) Thus, depending on the intended assay, a balance can be met between electrodeposition time and concentration detected. It is anticipated that the approximate cost of the paper device and reusable holder, at this early stage of development, is $1.22 and $1.88, respectively.

### CONCLUSIONS

In conclusion, a paper platform was developed that is ideal for POC applications because it is cheap (˜$1.22/device), user friendly (electrochemical detection and no washing steps), sensitive, portable, robust (AgNPs instead of enzymes), efficient (composite capture efficiency of 36±10% and charge collection efficiency of 13±2%), fast (completion of analysis in 4.6 min), and can detect low concentrations (767 fM). In addition, the proposed platform facilitates the timed introduction of reagents and it permits the integration of all the steps necessary for the automatic production of the signal, with the only requirements from the user being the injection of the sample and the slipping of a paper layer to activate the sensor. The proposed platform can be used to detect a myriad of analytes without having to change the source of the signal or the signal amplification method because the AgNPs and magnetic microbeads can be modified with various binding agents.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A method for detecting an analyte comprising:
(a) flowing fluid along a channel while applying a magnetic field to a region of the channel in electrochemical contact with a working electrode in order to accumulate the analyte conjugated to a metal particle and a magnetic particle in the region of the channel;
(b) oxidizing the metal particle to form metal ions; and
(c) electrochemically detecting the metal ions.

2. The method of claim 1, wherein step (a) comprises flowing fluid comprising the analyte conjugated to the metal particle and the magnetic particle along the channel, and applying the magnetic field to accumulate the analyte conjugated to the metal particle and the magnetic particle in the region of the channel.

3. The method of claim 1 or 2, further comprising interrupting fluid flow along the channel between steps (a) and (b).

4. The method of any of claims 1-3, wherein oxidizing the metal particle comprises contacting the metal particle with an oxidant.

5. The method of any of claims 1-4, wherein electrochemically detecting the metal ions comprises quantifying the concentration of the analyte.

6. The method of any of claims 1-5, wherein the analyte is selected from the group consisting of antibodies, peptides, proteins, polynucleotides, lipids, polysaccharides, small molecule organic compounds, pathogens, and combinations thereof.

7. The method of any of claims 1-6, wherein the analyte is bound to the metal particle by a recognition element.

8. The method of claim 7, wherein the recognition element comprises an antibody, polynucleotide, receptor, ligand, antigen, protein, small molecule organic compound, or combination thereof.

9. The method of any of claims 1-8, wherein the analyte is bound to the magnetic particle by a recognition element.

10. The method of claim 9, wherein the recognition element comprises an antibody, polynucleotide, receptor, ligand, antigen, protein, small molecule organic compound, or combination thereof.

11. The method of any of claims 1-10, wherein the analyte is bound to a first antibody and a second antibody, and wherein the metal particle is bound to the first antibody and the magnetic particle is bound to the second antibody.

12. The method of any of claims 1-10, wherein the analyte is bound to a first polynucleotide and a second polynucleotide, and wherein the metal particle is bound to the first polynucleotide and the magnetic particle is bound to the second polynucleotide.

13. The method of claim 12, wherein the analyte is a polynucleotide or a polynucleotide.

14. The method of any of claims 1-13, wherein the metal particle comprises a metal nanoparticle.

15. The method of any of claims 1-14, wherein the channel defines a path for fluid flow from a fluid inlet to a fluid outlet, and wherein the method further comprises injecting a sample comprising a molecule of interest into the fluid inlet.

16. The method of claim 15, wherein the molecule of interest is the analyte.

17. The method of claim 16, wherein the sample comprises the analyte conjugated to a metal particle and a magnetic particle.

18. The method of claim 15, wherein the analyte comprises a surrogate for the molecule of interest.

19. The method of claim 18, wherein the surrogate is conjugated to a fixed analyte support, the metal particle, the magnetic particle, or combinations thereof, and wherein the surrogate is displaced by the molecule of interest.

20. The method of any of claims 1-19, wherein the method further comprises flowing fluid along the channel while applying a magnetic field to the region of the channel in order to accumulate a second analyte conjugated to a second metal particle and a second magnetic particle in the region of the channel.

21. The method of claim 20, wherein the second metal particle comprises a different metal than the first metal particle.

22. The method of claim 20 or 21, wherein electrochemically detecting the metal ions comprises quantifying the concentration of the first analyte and the second analyte.

23. The method of any of claims 20-22, wherein electrochemically detecting the metal ions comprises quantifying the ratio of the first analyte to the second analyte.

24. A device comprising:
a channel defining a path for fluid flow from a fluid inlet to a fluid outlet;
a working electrode positioned in electrochemical contact with a region of the channel; and
a magnet configured to apply a magnetic field to the region of the channel.

25. The device of claim 24, further comprising an engageable platform;
wherein the engageable platform can be translocated from a retracted position to a deployed position;
wherein in the retracted position the engageable platform is fluidly independent from the channel; and
wherein in the deployed position the engageable platform is in fluid contact with the region of the channel.

26. The device of claim 25, wherein when the engageable platform is in the deployed position, the path for fluid flow from the fluid inlet to the fluid outlet is interrupted, such that fluid cannot flow from the fluid inlet to the fluid outlet.

27. The device of claim 25 or 26, wherein the engageable platform comprises an oxidant.

28. The device of claim 27, wherein the oxidant comprises potassium permanganate.

29. The device of any of claims 24-28, wherein the device is paper based.

30. The device of any of claims 24-29, wherein the channel comprises a hollow channel

31. The device of any of claims 24-30, further comprising a second working electrode in electrochemical contact with a second region of the channel, and a second magnet configured to apply a magnetic field to the second region of the channel.

32. The device of any of claims 24-31, further comprising a counter electrode, a reference electrode, or combinations thereof in electrochemical contact with the channel.

33. A device comprising:
a first layer comprising a top surface, a bottom surface, a fluid inlet defining a path for fluid flow from the top surface of the first layer to the bottom surface of the first layer, a fluid outlet defining a path for fluid flow from the bottom surface of the first layer to the top surface of the first layer, and a working electrode disposed on the bottom surface of the first layer;
a second layer, comprising a top surface, a bottom surface, a hydrophobic boundary defining a channel for fluid flow within the second layer, and a port defining a path for fluid flow from the bottom surface of the second layer to the top surface of the second layer;
a third layer, comprising a top surface, a bottom surface, a hydrophobic boundary defining a channel for fluid flow within the third layer, a port defining a path for fluid flow from the bottom surface of the third layer to the top surface of the third layer, and an engageable platform disposed within the third layer;
a fourth layer, comprising a top surface, a bottom surface, a channel defining a path for fluid flow within the fourth layer formed from a porous hydrophilic material, and a sink fluidly connected to the channel and formed from a porous hydrophilic material; and
a magnet;
wherein the bottom surface of the first layer is in contact with the top surface of the second layer, the bottom surface of the second layer is in contact with the top surface of the third layer, and the bottom surface of the third layer is in contact with the top surface of the fourth layer;
wherein the working electrode is in electrochemical contact with a region of the channel in the second layer;
wherein the magnet is aligned with the working electrode so as to apply a magnetic field within the region of the channel in the second layer in electrochemical contact with the working electrode;
wherein the first layer, the second layer, the third layer, and the fourth layer are aligned so as to form a path for fluid flow from the fluid inlet to the channel for fluid flow within the second layer, the channel for fluid flow within the third layer, and the channel within the fourth layer to the sink to the port in the third layer to the port in the second layer to the fluid outlet, and
wherein the third layer can be translocated from a retracted position to a deployed position such that:
when the third layer is in the retracted position, the port of the third layer fluidly connects the port of the second layer and the sink and the engageable platform is fluidly independent from the channel for fluid flow within the second layer; and
when the third layer is in the deployed position, the engageable platform is in fluid contact with the region of the channel for fluid flow within the second layer and the port of the third layer is not aligned with the port of the second layer and the sink such that the path for fluid flow from the sink to the port of the second layer is interrupted.

34. The device of claim 33, wherein the engagable platform comprises an oxidant.

35. The device of claim 34, wherein the oxidant comprises potassium permanganate.

36. The device of any of claims 33-35, wherein the device is paper based.

37. The device of any of claims 33-36, wherein the first layer, the second layer, and the fourth layer are fabricated from a single piece of paper that is folded to form the device.

38. The device of any of the claims 33-37, further comprising a second working electrode disposed on the bottom surface of the first layer and a second magnet,
wherein the second working electrode is in electrochemical contact with a second region of channel in the second layer, and
wherein the second magnet is aligned with the second working electrode so as to apply a magnetic field within the second region of the channel in the second layer in electrochemical contact with the second working electrode.

39. The device of any of claims 33-38, further comprising a counter electrode, a reference electrode, or combinations thereof disposed on the bottom surface of the first layer, wherein the counter electrode, the reference electrode, or the combinations thereof is in electrochemical with the channel in the second layer.

40. The device of any of claims 33-39, further comprising an indicator disposed on the sink, the port in the third layer, the port in the second layer, or combinations thereof.

41. The device of any of claims 33-40, wherein the fluid inlet comprises a reagent for the detection of a molecule of interest.

42. A method for detecting an analyte comprising:
(a) flowing fluid along a channel to accumulate the analyte conjugated to a metal particle in a region of the channel in electrochemical contact with a working electrode, wherein the analyte conjugated to the metal particle is accumulated in the region of the channel by a localization element;
(b) oxidizing the metal particle to form metal ions; and
(c) electrochemically detecting the metal ions,
wherein the localization element is selected from the group consisting of a physical barrier disposed in the region of the channel, a localization electrode configured to apply an electric field to the region of the channel, a magnet configured to apply a magnetic field the region of the channel, or a combination thereof.

43. The method of claim 42, wherein the analyte comprises an analyte conjugated to a metal particle and a magnetic particle, and wherein the localization element comprises a magnet configured to apply a magnetic field to the region of the channel.

44. The method of claim 43, wherein step (a) comprises flowing fluid comprising the analyte conjugated to the metal particle and the magnetic particle along the channel, and applying the magnetic field to accumulate the analyte conjugated to the metal particle and the magnetic particle in the region of the channel.

45. The method of claim 42, wherein the analyte is charged, and wherein the localization element comprises a localization electrode configured to apply an electric field to the region of the channel.

46. The method of claim 45, wherein step (a) comprises flowing fluid comprising the charged analyte conjugated to the metal particle along the channel, and applying electric field to accumulate the charged analyte conjugated to the metal particle in the region of the channel.

47. The method of claim 42, the localization element comprises a physical barrier disposed in the region of the channel, and wherein step (a) comprises flowing fluid comprising the analyte conjugated to the metal particle along the channel to contact the physical barrier such that the analyte accumulates in the region of the channel.

48. The method of any of claims 42-47, further comprising interrupting fluid flow along the channel between steps (a) and (b).

49. The method of any of claims 42-48, wherein oxidizing the metal particle comprises contacting the metal particle with an oxidant.

50. The method of any of claims 42-49, wherein electrochemically detecting the metal ions comprises quantifying the concentration of the analyte.

51. The method of any of claims 42-50, wherein the analyte is selected from the group consisting of antibodies, peptides, proteins, polynucleotides, lipids, polysaccharides, small molecule organic compounds, pathogens, and combinations thereof.

52. The method of any of claims 42-51, wherein the analyte is bound to the metal particle by a recognition element.

53. The method of claim 52, wherein the recognition element comprises an antibody, polynucleotide, receptor, ligand, antigen, or combination thereof.

54. The method of any of claims 42-53, wherein the metal particle comprises a metal nanoparticle.

55. The method of any of claims 42-54, wherein the channel defines a path for fluid flow from a fluid inlet to a fluid outlet, and wherein the method further comprises injecting a sample comprising a molecule of interest into the fluid inlet.

56. The method of claim 55, wherein the molecule of interest is the analyte.

57. The method of claim 56, wherein the sample comprises the analyte conjugated to a metal particle.

58. The method of claim 55, wherein the analyte comprises a surrogate for the molecule of interest.

59. The method of claim 58, wherein the surrogate is conjugated to a fixed analyte support and wherein the analyte is displaced by the molecule of interest.

60. The method of any of claims 42-59, wherein the method further comprises flowing fluid along a channel to accumulate a second analyte conjugated to a second metal particle in the region of the channel in electrochemical contact with the working electrode, wherein the second analyte conjugated to the second metal particle is accumulated in the region of the channel by a localization element.

61. The method of claim 60, wherein the second metal particle comprises a different metal than the first metal particle.

62. The method of claim 60 or 61, wherein electrochemically detecting the metal ions comprises quantifying the concentration of the first analyte and the second analyte.

63. The method of any of claims 60-62, wherein electrochemically detecting the metal ions comprises quantifying the ratio of the first analyte to the second analyte.

64. A device for the detection of an analyte conjugated to a metal particle comprising:
a channel defining a path for fluid flow from a fluid inlet to a fluid outlet;
a working electrode positioned in electrochemical contact with a region of the channel; and
a localization element configured to accumulate the analyte conjugated to the metal particle in the region of the channel in electrochemical contact with the working electrode,
wherein the localization element is selected from the group consisting of a physical barrier disposed in the region of the channel, a localization electrode configured to apply an electric field to the region of the channel, a magnet configured to apply a magnetic field the region of the channel, or a combination thereof.

65. The device of claim 64, further comprising an engageable platform;
wherein the engageable platform can be translocated from a retracted position to a deployed position;
wherein in the retracted position the engageable platform is fluidly independent from the channel; and
wherein in the deployed position the engageable platform is in fluid contact with the region of the channel.

66. The device of claim 65, wherein when the engageable platform is in the deployed position, the path for fluid flow from the fluid inlet to the fluid outlet is interrupted, such that fluid cannot flow from the fluid inlet to the fluid outlet.

67. The device of claim 65 or 66, wherein the engageable platform can be translocated from an incubation position to a retracted position to a deployed position,
wherein when the engageable platform is in the incubation position, the engageable platform is fluidly independent from the channel and the path for fluid flow from the fluid inlet to the fluid outlet is interrupted, such that fluid cannot flow from the fluid inlet to the fluid outlet.

68. The method of any of claims 65-67, wherein the engageable platform comprises an oxidant.

69. The device of claim 68, wherein the oxidant comprises potassium permanganate.

70. The device of any of claims 64-69, wherein the device is paper based.

71. The device of any of claims 64-70, wherein the channel comprises a hollow channel

72. The device of any of claims 64-71, further comprising a counter electrode, a reference electrode, or combinations thereof in electrochemical contact with the channel.
